(19) United States
(12) Patent Application Publication
(10) Pub. No.: US 2021/0052532 A1
(43) Pub. Date: Feb. 25, 2021

(54) COMPOSITIONS AND METHODS FOR TREATMENT OF INSULIN RESISTANCE

(71) Applicant: TEMPLE UNIVERSITY - OF THE COMMONWEALTH SYSTEM OF HIGHER EDUCATION, Philadelphia, PA (US)

(72) Inventors: Salim MERALI, Philadelphia, PA (US); Carlos A. BARRERO, Philadelphia, PA (US); Wayne E. CHILDERS, New Hope, PA (US); George C. MORTON, Collegeville, PA (US)

(73) Assignee: Temple University - Of The Commonwealth System Of Higher Education, Philadelphia, PA (US)

(21) Appl. No.: 16/977,684

(22) PCT Filed: Mar. 7, 2019
(86) PCT No.: PCT/US2019/021230

§ 371 Date: Sep. 2, 2020

Related U.S. Application Data
(60) Provisional application No. 62/639,880, filed on Mar. 7, 2018.

(51) Int. Cl. A61K 31/198

(52) U.S. Cl. CPC A61K 31/198; A61K 45/06

(57) ABSTRACT

In certain embodiments, this disclosure relates to methods of treating or preventing type 2 diabetes, pre-diabetes and conditions characterized by an increase in the levels of A1C, glucose, insulin, homeostasis model of assessment of insulin resistance HOMA-IR), oxidative stress in adipose tissue, and earbonvlation of GLUT4 comprising administering an effective amount of a compound of Formula I-III as described herein, to a subject in need thereof.

### CROSS-REFERENCE TO RELATED APPLICATION

This application claims the benefit of U.S. Provisional application No. 62/639,880, filed Mar. 7, 2018, the entire disclosure of which is incorporated herein by reference.

### SEQUENCE LISTING

The instant application contains a Sequence Listing which has been submitted in ASCII format via EFS-Web and is hereby incorporated by reference in its entirety. Said ASCII copy, created on Mar. 1, 2019 is named 035926_0501_00_WO_587256_ST25.txt and is 875 bytes in size.

### SUMMARY OF THE INVENTION

In embodiments, this disclosure relates to methods of treating or preventing type 2 diabetes, pre-diabetes and conditions characterized by an increase in the levels of A1C, glucose, insulin, homeostasis model of assessment of insulin resistance (HOMA-IR), oxidative stress in adipose tissue, and carbonylation of GLUT4 comprising administering an effective amount of a compound of Formula I-III as described herein, to a subject in need thereof.

### DESCRIPTION OF THE DRAWINGS

FIG. 1 demonstrated the stoichiometry of GLUT4 carbonylation in adipose tissue from obese non-diabetic (n=4), obese pre-diabetic (n=4), and obese diabetic (n=12) subjects (*p<0.005).

FIG. 2 provides a graphical depiction of the correlation between adipose tissue GLUT4 carbonylation and HbA1C.

FIG. 3 provides typical MRM data showing an increase in the transitions of HNE-induced K264-HNE adduct (left panel) which were used to calculate the amounts of carbonylated GLUT4 (right panel). Four transitions of the GLUT4 peptide found in humans are shown. The sequence of the carbonylated GLUT4 peptide LTGWADVSGVLAELKDEK-4HNE (SEQ ID NO:1) is depicted.

FIG. 4 graphically depicts glucose transport impairment in 3T3-L1 cells when exposed to 4-HNE (20 μM) and/or H₂O₂ (100 μM) for 4 hr.

### DETAILED DESCRIPTION

In Type 1 diabetes, also known as insulin-dependent diabetes mellitus (IDDM), or juvenile diabetes, the pancreas produces little or no insulin. Type 1 diabetes is believed to result in part from the autoimmune attack on the insulin producing beta-cells of the pancreas.

Type 2 diabetes mellitus (T2DM), also known as Non-Insulin Dependent Diabetes Mellitus (NIDDM), or adult-onset diabetes, is mostly caused by insulin resistance and eventually results in beta-cell exhaustion, leading to beta-cell destruction. Insulin resistance is associated with impairment of peripheral tissue response to insulin. T2DM is primarily due to obesity and insufficient exercise in people who are genetically predisposed. It makes up about 90% of cases of diabetes. Rates of T2DM have increased markedly since 1960 in parallel with obesity. It is believed to afflict approximately 18.2 million people in the US. T2DM typically begins in middle or older age. However, as a result of the obesity epidemic, substantially younger patients are diagnosed with this condition. Type 2 diabetes is associated with a ten-year-shorter life expectancy.

Insulin resistance is generally regarded as a pathological condition in which cells fail to respond to the normal actions of the hormone insulin. When the body produces insulin under conditions of insulin resistance, the cells in the body are resistant to the insulin and are unable to use it as effectively, leading to high blood sugar.

In the early stage of T2DM, the predominant abnormality is reduced insulin sensitivity and is commonly referred to as prediabetes. At this stage hyperglycemia can be reversed by a variety of measures and medications known in the art. In reaction to increasing insulin resistance, beta-cells are forced to produce more insulin, or are triggered to proliferate and/or granulate, producing even more insulin. The overproduction of insulin or over activity of beta-cells can then lead to beta-cell exhaustion, leading to destruction of the beta-cell population. The pancreas can thus no longer provide adequate levels of insulin, resulting in elevated levels of glucose in the blood. Ultimately, overt hyperglycemia and hyperlipidemia occur, leading to the devastating long-term complications associated with diabetes, including cardiovascular disease, renal failure, and blindness.

Insulin resistance is present in almost all obese individuals. Obesity-linked insulin resistance greatly increases the risk for T2DM, hypertension, dyslipidemia, and nonalcoholic fatty liver disease, together known as the metabolic or insulin resistance syndrome.

Insulin resistance and T2DM are associated with an increased risk of heart attacks, strokes, amputation, diabetic retinopathy, and kidney failure. For extreme cases, circulation in the limbs is affected, potentially requiring amputation. Loss of hearing, eyesight, and cognitive ability has also been linked to these conditions.

Identifying and treating the initial changes that occur due to overnutrition will prevent prediabetes from progressing into T2DM. In overnutrition, excessive glucose is consumed and a large amount of glucose is metabolized via glycolysis and the TCA cycle leading to increased NADH and FADH₂ production in the mitochondrial electron transport chain and increased reactive oxygen species (ROS). When the generation of ROS exceeds their detoxification, oxidative stress occurs. Oxidative stress may cause reversible or irreversible changes in proteins. Reversible changes occur in cysteine residues and can be repaired by antioxidant proteins. On the other hand, oxidative stress can directly or indirectly induce irreversible damage to the proteins by formation of reactive carbonyl groups, mainly aldehydes and ketones. Direct protein carbonylation of lysine or arginine residues occurs through a Fenton reaction of metal cations with hydrogen peroxide, forming glutamic semialdehyde. Indirect carbonylation can occur by reactive α,β-unsaturated aldehydes, which are products of oxidative modification of polyunsaturated fatty acids (PUFA).

The most common reactive aldehyde is 4-hydroxynonenal (4-HNE). 4-HNE reacts with cysteine, lysine, and histidine residues of proteins via Michael addition and Schiff base formation. The introduction of carbonyl derivatives (i.e. aldehydes and ketones) alters the conformation of the polypeptide chain, resulting in the partial or total inactivation of proteins. Because protein carbonylation is an irreversible process, it is deleterious to the cells. 4-HNE increases have been reported in T2DM and in the liver of diabetic rats.

In a study reported in 2015, healthy men were fed ˜6000 kcal/day of the common U.S. diet [˜50% carbohydrate (CHO), ˜35% fat, and ˜15% protein] for 1 week. The diet produced a rapid weight gain of 3.5 kg and the rapid onset (after 2 to 3 days) of systemic and adipose tissue insulin resistance and oxidative stress but no inflammatory or ER stress. In adipose tissue, the oxidative stress was associated with several GLUT4 posttranslational modifications, including extensive GLUT4 carbonylation as well as adduction of HNE and glutamic semialdehyde in close proximity to the glucose transport channel. GLUT4 is the major insulin-facilitated glucose transporter in adipose tissue. Carbonylation typically causes protein cross-linking and loss or alteration of protein function and can target the affected proteins for selective degradation by the 26S proteasome.

Notwithstanding these advances, what is still needed are therapeutic agents for the prevention and treatment of insulin resistance, particularly in obese patients who typically suffer from insulin resistance or are most susceptible to the development of insulin resistance, and ultimately, type 2 diabetes. The embodiments described herein provide compounds which treat or prevent type 2 diabetes, pre-diabetes and conditions characterized by an increase in the levels of A1C, glucose, insulin, homeostasis model of assessment of insulin resistance (HOMA-IR), oxidative stress in adipose tissue, and carbonylation of GLUT4.

Unless defined otherwise, all technical and scientific terms used herein have the same meaning as commonly understood by one of ordinary skill in the art to which the invention pertains. In describing and claiming the present invention, the following terminology will be used. It is also to be understood that the terminology used herein is for the purpose of describing particular embodiments only, and is not intended to be limiting.

Unless the context requires otherwise, throughout the specification and claims which follow, the word “comprise” and variations thereof, such as, “comprises,” “comprising” “including,” “containing,” or “characterized by,” are to be construed in an open, inclusive sense, that is, as “including, but not limited to” and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The articles “a” and “an” are used herein to refer to one or to more than one (i.e., to at least one) of the grammatical object of the article. By way of example, “an element” means one element or mom than one element. Thus, recitation of “a cell”, for example, includes a plurality of the cells of the same type.

The word “about” when immediately preceding a numerical value means a range of plus or minus 20% of that value, or plus or minus 10% of that value, e.g, “about 50” means 45 to 55, “about 25,000” means 22,500 to 27,500, etc, more preferably plus or minus 5% of that value, more preferably plus or minus 1% of that value, and still more preferably plus or minus 0.1% of that value, unless the context of the disclosure indicates otherwise, or is inconsistent with such an interpretation. Furthermore, the phrases “less than about” a value or “greater than about” a value should be understood in view of the definition of the term “about” provided herein.

The terms “administer,” “administering” or “administration” as used herein refer to either directly administering a compound or pharmaceutically acceptable salt of the compound or a composition to a subject.

The term “alkyl”, by itself or as part of another substitunt means, unless otherwise stated, a straight or branched chain hydrocarbyl having the designated number of carbon atoms (i.e., C₁-C₆ means one to six carbons). Examples include: methyl, ethyl, propyl, isopropyl, butyl, isobutyl, tert-butyl, pentyl, neopentyl, and hexyl. Most preferred is (C₁-C₆)alkyl, more preferably (C₁-C₃)alkyl, particularly methyl and ethyl.

The term “alkenyl” employed alone or in combination with other terms, means, unless otherwise stated, a straight chain or branched chain hydrocarbyl having the stated number of carbon atoms, and containing one or more double bonds. Examples include ethenyl (vinyl), propenyl (allyl), crotyl, isopentenyl, butadienyl, 1,3-pentadienyl, and 1,4-pentadienyl. A functional group representing an alkenyl is exemplified by —CH₂—CH═CH₂—.

The term “alkynyl” employed alone or in combination with other terms, means, unless otherwise stated, a straight chain or branched chain hydrocarbyl having the stated number of carbon atoms, and containing one or more triple bonds.

The term “alkoxy” employed alone or in combination with other terms means, unless otherwise stated, an alkyl group, as defined above, connected to the rest of the molecule via an oxygen atom, such as, for example, methoxy, ethoxy, 1-propoxy, 2-propoxy (isopropoxy) and the higher homologs and isomers. The alkyl portion of the alkoxy group can have a designated number of carbon atoms as defined for alkyl groups above. Preferred are (C₁-C₆)alkoxy, more preferably (C₁-C₃)alkoxy, particularly methoxy and ethoxy.

The term “aromatic” refers to a carbocycle or heterocycle having one or more polyunsaturated rings having aromatic character (i.e. having (4n+2) delocalized π(pi) electrons where n is an integer).

The term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “aralkyl” group refers to an alkyl group substituted with an aryl group.

As used herein, the term “combination with” when used to describe administration with an additional treatment means that the agent may be administered prior to, together with, or after the additional treatment, or a combination thereof.

An “effective amount” or “therapeutically effective amount” as used herein, means an amount which provides the indicated therapeutic or prophylactic benefit, i.e., an amount that results in the treatment and/or prevention of insulin resistance and/or an increase in insulin sensitivity, or treatment and/or prevention if insulin resistance disorder. It is understood, however, that the full therapeutic effect does not necessarily occur by administration of one dose, and may occur only after administration of a series of doses. Thus, an effective amount may be administered in one or more administrations. In the context of therapeutic or prophylactic applications, the amount of active agent administered to the subject will depend on the type and severity of the disease or condition and on the characteristics of the subject, such as general health, age, sex, body weight and tolerance to drugs. It will also depend on the degree, severity and type of disease or condition. The skilled artisan will be able to determine appropriate dosages depending on these and other factors. The compounds of Formula I-III can also be administered in combination with one or more additional therapeutic compounds.

The terms “halo” or “halogen” by themselves or as part of another substituent mean, unless otherwise stated, a fluorine, chlorine, bromine, or iodine atom. Preferably, a halogen includes fluorine, chlorine, or bromine, more preferably, fluorine or chlorine.

The term “heteroaralkyl” group refers to an alkyl group substituted with a heteroaryl group.

The term “heterocycle” or “heterocyclyl” or “heterocyclic” by itself or as part of another substituent means, unless otherwise stated, an unsubstituted or substituted, mono- or multi-cyclic heterocyclic ring system which consists of carbon atoms and at least one heteroatom selected from the group consisting of N, O, and S. The heterocycle typically contains from five to ten ring atoms. The heterocyclic system may be attached to another atom, unless otherwise stated, at any heteroatom or carbon atom of the heterocyclic system which affords a structural isomer.

The term “heteroaryl” or “heteroaromatic” refers to a heterocycle having aromatic character.

The term “hydrocarbyl”, by itself or as part of another substituent means, unless otherwise stated, a straight or branched chain hydrocarbon having the number of carbon atoms designated (i.e. C₁-C₆ means one to six carbons). Examples include: methyl, ethyl, propyl, isopropyl, butyl, isobutyl, tert-butyl, pentyl, neopentyl, and hexyl. Most preferred is (C₁-C₆) alkyl, more preferably (C₁-C₃) particularly methyl and ethyl. The term “unsaturated hydrocarbyl” means a hydrocarbyl that contains at least one double or triple bond.

As used herein, “individual” or “patient” or “subject” (as in the subject of the treatment) means both mammals and non-mammals. Mammals include, for example, humans; non-human primates, e.g. apes and monkeys; dogs; cats; cattle; horses; sheep; and goats. Non-mammals include, for example, fish and birds. The individual is, in one embodiment, a human being. In another embodiment, the individual is a dog.

The term “insulin resistance” has its common meaning in the art. Insulin resistance is a physiological condition where the natural hormone insulin becomes less effective at lowering blood sugars. The resulting increase in blood glucose may raise levels outside the normal range and cause adverse health effects such as metabolic syndrome, dyslipidemia and subsequently type 2 diabetes mellitus.

An “insulin resistance disorder” refers to any disease or condition that is caused by or contributed to by insulin resistance. Examples include: diabetes, obesity, metabolic syndrome, insulin resistance, insulin resistance syndromes, syndrome X, high blood pressure, hypertension, high blood cholesterol, dyslipidemia, hyperlipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

The term “haloalkyl” means an alkyl group wherein at least one hydrogen atom is replaced by a halogen atom. The term “perhaloalkyl” means a haloalkyl group wherein all the hydrogen atoms are replaced by halogen atoms. A preferred perhaloalkyl is perfluoroalkyl, particularly —(C₁-C₆)perfluoroalkyl; more preferred is —(C₁-C₃)perfluoroalkyl; most preferred is —CF₃.

The term “haloalkoxy” means an alkoxy group wherein at least one hydrogen atom is replaced by a halogen atom. The term “perhaloalkoxy” means a haloalkoxy group wherein all the hydrogen atoms are replaced by halogen atoms. A preferred perhaloalkoxy is perfluoroalkoxy, particularly —(C₁-C₆)perfluoroalkoxy; more preferred is —(C₁-C₃)perfluoroalkoxy; most preferred is —OCF₃.

As used herein, the term “pharmaceutically acceptable” refers to a formulation of a compound that does not significantly abrogate the biological activity, a pharmacological activity and/or other properties of the compound when the formulated compound is administered to a patient. In certain embodiments, a pharmaceutically acceptable formulation does not cause significant irritation to a patient.

“Pharmaceutically acceptable carrier” means any carrier, diluent or excipient which is compatible with the other ingredients of the formulation and not deleterious to the recipient.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. The salts can be prepared in situ during the isolation and purification of the compounds of the disclosure, or separately by reacting the free base or free acid of a compound of the disclosure with a suitable acid or base, respectively. Examples of pharmaceutically acceptable, nontoxic acid addition salts are salts of an amino group formed with inorganic acids such as hydrochloric acid, hydrobromic acid, hydroiodic acid, carbonic acid, phosphoric acid, sulfuric acid and pembloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, anthranilic, ascorbate, aspartate, benzenesulfonate, benzoate, bisulfate, borate, butyrate, camphorate, camphorsulfonate, citrate, cyclohexylaminosulfonic, cyclopentanepropionate, digluconate, dodecylsulfate, ethanesulfonate, formate, fumarate, furoic, galactaric, galacturonic, glucoheptonate, glycerophosphate, glycolic, gluconate, glucuronic, glutamic, hemisulfate, heptanoate, hexanoate, hydroiodide, 4-hydroxybenzoic, β-hydroxybutyric, 2-hydroxyethanesulfonate, isethionic, lactobionate, lactate, laurate, lauryl sulfate, malate, maleate, malonate, mandelic, methane-sulfonate, mucic, 2-naphthalenesulfonate, nicotinate, nitrate, oleate, oxalate, palmitate, pamoate, pantothenic, pectinate, persulfate, phenylacetic, 3-phenylpropionate, phosphate, picrate, pivalate, propionate, pyruvic, salicylic, stearate, succinate, sulfate, sulfanilic, tartrate, thiocyanate, p-toluenesulfonate, trifluoromethanesulfonic, undecanoate, valerate salts, and the like. Representative alkali, alkaline earth metal salts, or transition metal salts include sodium, lithium, potassium, calcium, magnesium, zinc and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkylsulfonate and aryl sulfonate. Other pharmaceutically acceptable base addition salts include N,N′-dibenzylethylenediamine, chloroprocaine, choline, diethanolamine, ethylenediamine, tromethamine, meglumine (N-methylglucamine) and procaine.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. Substituents may include, for example, one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, alkylsilyl, cycloalkyl, cycloalkylalkyl, heterocycloalkyl, heteroaryl, alkenyl, alkynyl, aryl, and amino groups. Substituents comprising carbon chains preferably contain 1-6, more preferably 1-3, most preferably 1-2, carbon atoms.

To “treat” a disease as the term is used herein, means to reduce the frequency or severity of at least one sign or symptom of a disease or disorder experienced by a subject. Treating may include the postponement of further disease progression, or reduction in the severity of symptoms that have or are expected to develop, ameliorating existing symptoms and preventing additional symptoms.

The term “unit dosage form” refers to physically discrete units suitable as a unitary dosage for human subjects and other mammals, each unit containing a predetermined quantity of active material calculated to produce the desired therapeutic effect, in association with a suitable pharmaceutical excipient.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Embodiments of the present invention are described below. It is, however, expressly noted that the present invention is not limited to these embodiments, but rather the intention is that modifications that are apparent to the person skilled in the art and equivalents thereof are also included.

Certain conditions such as overnutrition can lead to oxidative stress, and the generation of reactive aldehydes such as 4-HNE, which react with cysteine, lysine and histidine residues of proteins via Michael addition and Schiff base formation. 4-HNE can form HNE-Michaels adducts on GLUT4, the major insulin-facilitated glucose transporter in adipose tissue. 3T3-L1 adipocytes retrovirally transduced to overexpress the GLUT4-SNAP protein formed a K264-HNE GLUT4 adduct upon treatment with 4-HNE. The same K264-HNE GLUT4 adduct is elevated in the fat tissue of human pre-diabetic and diabetic individuals.

HNE-adduction leads to loss of GLUT-4 function, and development of adipocyte insulin resistance, as indicated by the reduction of adipocyte glucose uptake upon insulin stimulation.

Compounds of Formula I-III have been found to overcome adipocyte glucose uptake impairment by restoring insulin sensitivity. Without wishing to be bound by any theory, compounds of Formula I-III form adducts with reactive aldehydes such as 4-HNE, thereby diverting 4-HNE from damaging proteins such as GLUT-4, by carbonylation. (S)-2-amino-6-((3-aminopropyl)amino)hexanoic acid dihydrochloride, a compound of Formula I-III, forms an adduct with 4-HNE, thereby diverting 4-HNE from damaging GLUT-4 by carbonylation. It has the effect of reversing overnutrition-induced glucose uptake impairment. Restoration of GLUT-4 function results in enhancement or restoration of adipocyte insulin sensitivity and the resumption or enhancement of glucose uptake.

Impaired glucose tolerance as measured by glucose tolerance tests is dramatically improved in comparison to the only moderate glucose tolerance-improving effect of pioglitazone. The compounds of formula I-III are also better in reducing impaired glucose tolerance than metformin. Metformin is a first-line medication for the treatment of type 2 diabetes.

Compounds of Formula I-III can be used to treat both pre-diabetes, and type 2 diabetes wherein the diabetic phenotype has been established. The compounds are believed effective in counteracting glucose uptake impairment in cells induced by overnutrition.

The compounds of Formula I-III are administered to increase insulin sensitivity and/or reduce insulin resistance in subjects in need of such treatment.

According to the present invention, any of the pathologies flowing from reduced insulin sensitivity (or insulin resistance) may be treated. The compounds of Formula I-III are thus useful for treating any condition associated with the loss of the relevant target cell's sensitivity to regulation by insulin. The compounds of Formula I-III are thus believed useful in the treatment of insulin resistance disorders.

In addition to pathological conditions associated with insulin resistance, the compounds of Formula I-III may be administered for treatment of conditions of low insulin production, e.g. cases of IDDM where some finite level of insulin production remains, albeit at reduced amounts.

### Genus

In certain embodiments of the present invention, compounds have structural Formula I:

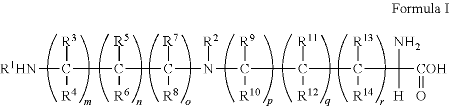

wherein:
R¹ is selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —NH(C₁-C₆)alkyl, —N[(C₁-C₆)alkyl)]₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
R² is selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
R³, R⁴, R⁷, R⁸, R⁹, R¹⁰, R¹³, and R¹⁴ are independently selected from the group consisting of hydrogen and —(C₁-C₆)alkyl;
R⁵ and R⁶ are independently selected from the group consisting of hydrogen, —(C₁-C₆)alkyl and —OH, provided that both R⁵ and R⁶ cannot be —OH;
R¹¹ and R¹² are independently selected from the group consisting of hydrogen, —(C₁-C₆)alkyl and —OH, provided that both R¹¹ and R¹² cannot be —OH;
m is 1, 2, 3 or 4;
n is 0, 1, 2, 3 or 4;
o is 0, 1, 2, 3 or 4;
p is 1, 2, 3 or 4;
q is 0, 1, 2, 3 or 4; and
r is 0, 1, 2, 3 or 4.

In certain embodiments of compounds of Formula I, the proviso applies that when the sum of m, n, and o is 3 and the sum of p, q, and r is 3 then R¹, R², R³, R⁴, R⁵, R⁶, R⁷, R⁸, R⁹, R¹⁰, R¹¹, R¹², R¹³, and R¹⁴ are not all H.

In certain embodiments of compounds of Formula I, R¹ and/or R² are selected from perhalo(C₁-C₆)alkyl and perhalo(C₁-C₆)alkoxy.

In certain embodiments of compounds of Formula I, R¹ is selected from hydrogen and —(C₁-C₈)alkyl. In certain embodiments, R² is selected from hydrogen or —(C₁-C₈)alkyl. In certain embodiments, R¹ and R² are independently selected from hydrogen and —(C₁-C₈)alkyl. In the aforementioned embodiments, the —(C₁-C₈)alky is preferably —(C₁-C₆)alkyl, more preferably —(C₁-C₃)alkyl, more preferably methyl or ethyl. In certain embodiments, R¹ and R² are hydrogen.

In certain embodiments of compounds of Formula I, each of R³, R⁴, R⁵, R⁶, R⁷, and R⁸ is independently selected from hydrogen and —(C₁-C₈)alkyl. The —(C₁-C₈)alkyl is preferably —(C₁-C₆)alkyl, more preferably —(C₁-C₃)alkyl, more preferably methyl or ethyl. In certain embodiments, R³, R⁴, R⁵, R⁶, R⁷, and R⁸ are hydrogen.

In certain embodiments of compounds of Formula I, each of R⁹, R¹⁰, R¹¹, R¹², R¹³, and R¹⁴ is independently selected from hydrogen and —(C₁-C₈)alkyl. The —(C₁-C₈)alkyl is preferably —(C₁-C₆)alkyl, more preferably —(C₁-C₃)alkyl, more preferably methyl or ethyl. In certain embodiments, R⁹, R¹⁰, R¹¹, R¹², R¹³, and R¹⁴ are hydrogen.

In certain embodiments of compounds of Formula I, each of R³ through R¹⁴ are independently selected from hydrogen and —(C₁-C₈)alkyl, according to the above schemes. In certain embodiments, R³ through R¹⁴ are hydrogen.

In some embodiments of compounds of Formula I, the sum of m+n+o is in the range of from 2 to 10, 9, 8, 7, 6, 5, 4 or 3; in the range of from 3 to 10, 9, 8, 7, 6, 5 or 4; or in the range of from 4 to 10, 9, 8, 7, 6 or 5. In some embodiments, the sum of m+n+o is 12, 11, 10, 9, 8, 7, 6, 5, 4, 3 or 2.

In some embodiments of the aforesaid embodiments of compounds of Formula I defining sums of m+n+o and/or defining sums of p+q+r, each of R³ through R¹⁴ are independently selected from hydrogen and —(C₁-C₈)alkyl. In certain embodiments, R³ through R¹⁴ are hydrogen.

In certain preferred embodiments of a compound of Formula I, m is 3; p is 4; and each of n, o, q and r is zero. In certain such embodiments, R³, R⁴, R⁹, and R¹⁰ are independently selected from hydrogen and —(C₁-C₈)alkyl, preferably hydrogen. In certain such embodiments, R¹ and R² may be independently selected from hydrogen and —(C₁-C₈)alkyl, preferably hydrogen.

In certain embodiments of the present invention, compounds have structural Formula II:

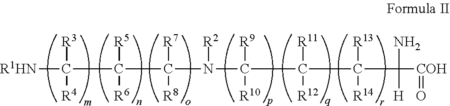

wherein
R¹, R², R³, R⁴, R⁵, R⁶, R⁷, R⁸, R⁹, R¹⁰, R¹¹, R¹², R¹³, R¹⁴, m, n, o, p, q, and r defined as above for Formula I, provided:
   (i) when the sum of p+q+r is 1, then sum of m+n+o is 5 or greater,
   (ii) when the sum of p+q+r is 2, the sum of m+n+o is 5 or greater,
   (iii) when the sum of p+q+r is 3, the sum of m+n+o is 3, or is greater,
   (iv) when the sum of p+q+r is 4, the sum of m+n+o is either 3, or is 6 or greater; or a variant thereof.

In certain embodiments of compounds of Formula II, when the sum of p+q+r is 2, the sum of m+n+o is 6 or greater, 7 or greater, 8 or greater, 9 or greater or 10 or greater.

In certain embodiments of compounds of Formula II, when the sum of p+q+r is 3, the sum of m+n+o is 5 or greater, 6 or greater, 7 or greater, 8 or greater, 9 or greater or 10 or greater.

In certain embodiments of compounds of Formula II, when the sum of p+q+r is 4, the sum of m+n+o is 7 or greater, 8 or greater, 9 or greater or 10 or greater.

In embodiments of compounds of Formula II, when the sum of p+q+r is 4, the sum of m+n+o is 3 and R¹, R², R³, R⁴, R⁵, R⁶, R⁷, R⁸, R⁹, R¹⁰, R¹¹, R¹², R¹³, and R¹⁴ are not all H.

In certain embodiments of compounds of Formula II, where the sum of p+q+r is 1, then the sum of m+n+o is 5 or greater, 6 or greater, 7 or greater, 8 or greater, 9 or greater or 10 or greater.

In certain embodiments of the compounds of Formula II, R¹ is selected from hydrogen and —(C₁-C₈)alkyl. In certain embodiments, R² is selected from hydrogen or —(C₁-C₈)alkyl. In certain embodiments, R¹ and R² are independently selected from hydrogen and —(C₁-C₈)alkyl. In the aforementioned embodiments, the —(C₁-C₈)alkyl is preferably —(C₁-C₆)alkyl, more preferably —(C₁-C₃)alkyl, more preferably methyl or ethyl. In certain embodiments, R¹ and R² are hydrogen.

In certain embodiments of the compounds of Formula II, each of R³, R⁴, R⁵, R⁶, R⁷, and R⁸ is independently selected from hydrogen and —(C₁-C₈)alkyl. The —(C₁-C₈)alkyl is preferably —(C₁-C₆)alkyl, more preferably —(C₁-C₃)alkyl, more preferably methyl or ethyl. In certain embodiments, R³, R⁴, R⁵, R⁶, R⁷, and R⁸ are hydrogen.

In certain embodiments of the compounds of Formula II, each of R⁹, R¹⁰, R¹¹, R¹², R¹³, and R¹⁴ is independently selected from hydrogen and —(C₁-C₈)alkyl. The —(C₁-C₈)alkyl is preferably —(C₁-C₆)alkyl, more preferably —(C₁-C₃)alkyl, more preferably methyl or ethyl. In certain embodiments, R⁹, R¹⁰, R¹¹, R¹², R¹³, and R¹⁴ are hydrogen.

In certain embodiments of the compounds of Formula II, each of R³ through R¹⁴ are independently selected from hydrogen and —(C₁-C₈)alkyl, according to the above schemes. In certain embodiments, R³ through R¹⁴ are hydrogen.

In some embodiments of the novel compounds of Formula I, the sum of m+n+o is in the range of from 2 to 10, 9, 8, 7, 6, 5, 4 or 3; in the range of from 3 to 10, 9, 8, 7, 6, 5 or 4; or in the range of from 4 to 10, 9, 8, 7, 6 or 5. In some embodiments, the sum of m+n+o is 12, 11, 10, 9, 8, 7, 6, 5, 4, 3 or 2. The selection of the sum of m+n+o is subject to provisos (i), (ii), (iii) and (iv) above, for Formula II.

In some embodiments of compounds of Formula II, the sum of p+q+r is in the range of from 1 to 10, 9, 8, 7, 6, 5, 4 or 2; in the range of from 2 to 10, 9, 8, 7, 6, 5, 4 or 3; in the range of from 3 to 10, 9, 8, 7, 6, 5 or 4; or in the range of from 4 to 10, 9, 8, 7, 6 or 5. In some embodiments, the sum of p+q+r is 12, 11, 10, 9, 8, 7, 6, 5, 4, 3 or 2. The selection of the sum of p+q+r is subject to provisos (i), (ii), (iii) and (iv), above.

In some of the aforesaid embodiments of compounds of Formula II defining sums of m+n+o and/or defining sums of p+q+r, each of R³ through R¹⁴ are independently selected from hydrogen and —(C₁-C₈)alkyl. In certain embodiments, R³ through R¹⁴ are hydrogen.

In certain embodiments of the present invention, compounds have structural Formula III:

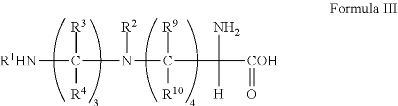

wherein each R³, each R⁴, each R⁹, and each R¹⁰ is independently selected from hydrogen and —(C₁-C₈)alkyl;

or a variant thereof.

In certain embodiments of compounds of Formula III, R³, R⁴, R⁹, and R¹⁰ am hydrogen.

In certain embodiments of compounds of Formula III, R¹ and R² are independently selected from the group consisting of hydrogen and —(C₁-C₈)alkyl, and are preferably hydrogen.

### Lead Compounds

The invention is further illustrated by the following examples of compounds of Formula I. In embodiments each of the following the compounds of Formula I are the L-isomer substantially free of the D-isomer.

(S)-2-amino-6-((6-aminohexyl)amino)hexanoic acid or a variant thereof.

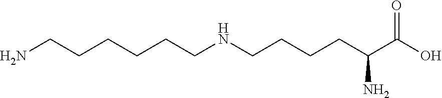 [SMILES: NCCCCCCNCCCC[C@H](N)C(=O)O]

(S)-2-amino-5-((6-aminohexyl)amino)pentanoic acid or a variant thereof.

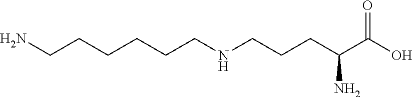 [SMILES: NCCCCCCNCCC[C@H](N)C(=O)O]

(S)-2-amino-5-((5-aminopentyl)amino)pentanoic acid or a variant thereof.

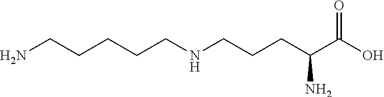 [SMILES: NCCCCCNCCC[C@H](N)C(=O)O]

The invention is further illustrated by the following examples of compounds of Formula III.

(S)-2-amino-6-((3-aminopropyl)amino)hexanoic acid or a variant thereof.

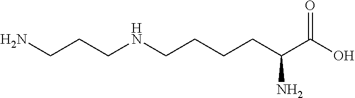 [SMILES: NCCCNCCCC[C@H](N)C(=O)O]

### General Synthetic Methods for Preparing Compounds

Compounds of Formula I may be prepared according to Schemes 1-16 wherein:

A is

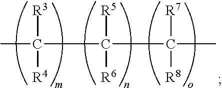

B is

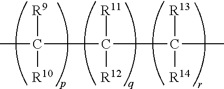

Compounds of Formula I may be prepared according to the general methods of Schemes 4-8 and 14-16. Certain compounds of Formula I, identified as having the structure of Formula Ia, may be prepared using the general methods shown in Schemes 1-3. Similarly, certain compounds of Formula I, identified as having the structure of Formula Ib, may be prepared using the general methods shown in Schemes 9-13. It may be appreciated that compounds of Formula Ia and Ib are compounds of Formula I wherein m is 1, and R³ and R are each hydrogen.

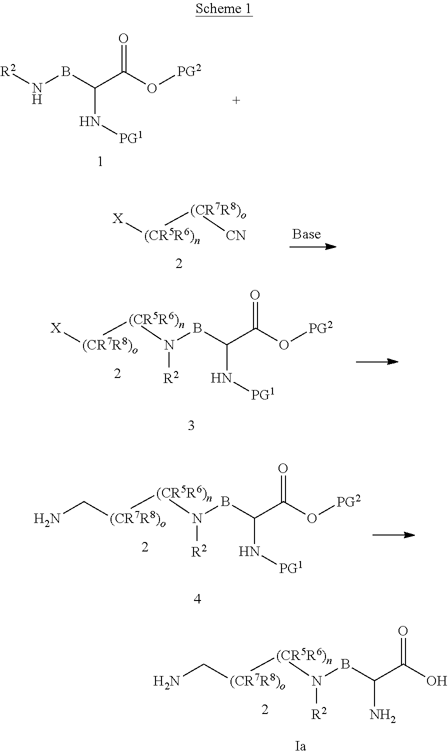

According to Scheme 1, a compound of the formula (1), a known compound or a compound prepared by known means wherein PG¹ is a protecting group selected, for example, from the group consisting of triphenylmethyl (trityl), tertbutyloxycarbonyl (BOC), 9-fluorenylmethyloxycarbonyl (FMOC), and carbobenzyloxy (Cbz) and PG² is selected, for example, from the group consisting of 9-fluorenylmethyl (Fm), C₁₋₆ alkyl and C₃₋₇ branched alkyl, is reacted with a compound of the formula (2), a known compound or compound prepared by known methods wherein X is a leaving group such as bromine, chlorine, iodine, methanesulfonate, tolylsulfonate, and the like, in the presence of a base such as trimethylamine, diisopropylethylamine, pyridine, potassium carbonate, cesium carbonate, sodium hydroxide, potassium hydroxide, sodium hydride, potassium hydride, lithium diisopropylamide, sodium hexamethyldisilazide, lithium hexamethyldisilazide, potassium t-butoxide, or sodium t-butoxide, and the like, in a solvent such as tetrahydrofuran, 1,4-dioxane, methylene chloride, methanol, ethanol, t-butanol, and the like, optionally with heating, optionally with microwave irradiation, to provide a compound of the formula (3).

According to Scheme 1, a compound of the formula (3) is then reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium, [1,1′-bis(diphenylphosphino) ferrocene]dichloropalladium, platinum on carbon, platinum on barium sulfate, platinum on celite, platinum on calcium carbonate, platinum on barium carbonate, platinum on silica, platinum on alumina, rhodium on carbon, rhodium on barium sulfate, rhodium on celite, rhodium on calcium carbonate, rhodium on barium carbonate, rhodium on silica, rhodium on alumina, and the like, in a solvent such as ethanol, methanol, tetrahydrofuran, 1,4-dioxane, ethyl acetate, benzene, toluene, cyclohexane, N,N-dimethylformamide, and the like, optionally with heating, optionally with microwave irradiation, to provide a compound of the formula (4).

According to Scheme 1, a compound of the formula (4) is then reacted with an acid such as acetic acid, trifluoroacetic acid, hydrochloric acid, sulfuric acid, trifluoromethanesulfonic acid, and the like, optionally in a solvent such as methylene chloride, tetrahydrofuran, 1,4-dioxane, ethanol, or methanol, and the like, optionally with heating, optionally with microwave irradiation, to provide a compound of the formula (Ia). Alternatively, a compound of the formula (4) is reacted with a base such as lithium hydroxide, sodium hydroxide, sodium carbonate, lithium carbonate, piperidine, pyridine, 2,6-lutidine, and the like, in a solvent such as methylene chloride, tetrahydrofuran, 1,4-dioxane, ethanol, or methanol, and the like, optionally with heating, optionally with microwave irradiation, to provide a compound of the formula (Ia). Alternatively, a compound of the formula (4) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, or methanol, and the like, optionally with heating, optionally with microwave irradiation, to provide a compound of the formula (Ia).

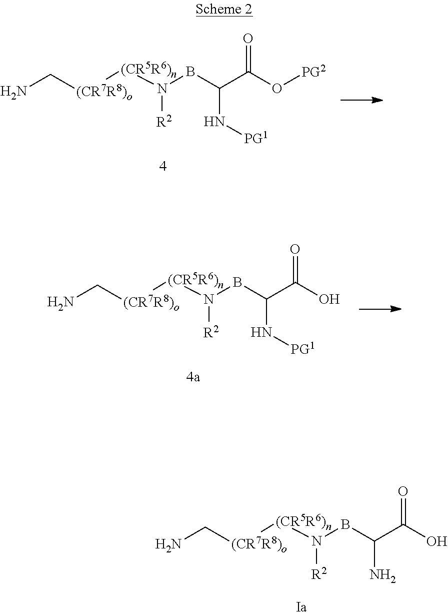

Alternatively, according to Scheme 2, a compound of the formula (4) is reacted with an acid such as acetic acid, trifluoroacetic acid, hydrochloric acid, sulfuric acid trifluoromethanesulfonic acid, and the like, optionally in a solvent such as methylene chloride, tetrahydrofuran, 1,4-dioxane, ethanol or methanol, and the like, optionally with heating, optionally with microwave irradiation to provide a compound of the formula (4a). Alternatively, according to Scheme 2, a compound of the formula (4) is reacted with a base such as lithium hydroxide, sodium hydroxide, sodium carbonate or lithium carbonate, and the like, in a solvent such as methylene chloride, tetrahydrofuran, 1,4-dioxane, ethanol or methanol, and the like, optionally with heating, optionally with microwave irradiation to provide a compound of the formula (4a).

According to Scheme 2, a compound of the formula (4a) is then reacted with a base such as piperidine, pyridine or 2,6-lutidine, and the like, in a solvent such as methylene chloride, tetrahydrofuran, 1,4-dioxane, ethanol or methanol, and the like, optionally with heating, optionally with microwave irradiation to provide a compound of the formula (Ia). Alternatively, a compound of the formula (4a) is reacted, according to Scheme 2, with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (Ia). Alternatively, a compound of the formula (4a) is reacted with an acid such as acetic acid, trifluoroacetic acid, hydrochloric acid, sulfuric acid, and the like, optionally in a solvent such as methylene chloride, tetrahydrofuran, 1,4-dioxane, ethanol, methanol, and the like, optionally with heating, optionally with microwave irradiation to provide a compound of the formula (Ia).

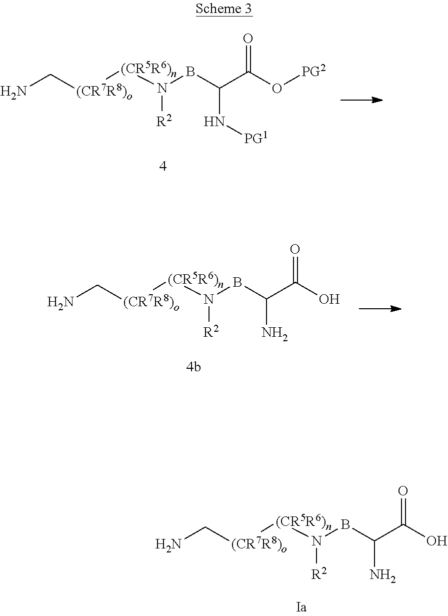

According to Scheme 3, a compound of the formula (4) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (4b). Alternatively, according to Scheme 3, a compound of the formula (4) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (4b).

According to Scheme 3, a compound of the formula (4b) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (Ia). Alternatively, according to Scheme 3, a compound of the formula (4b) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphospbino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (I). Alternatively, according to Scheme 3, a compound of the formula (4b) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (Ia).

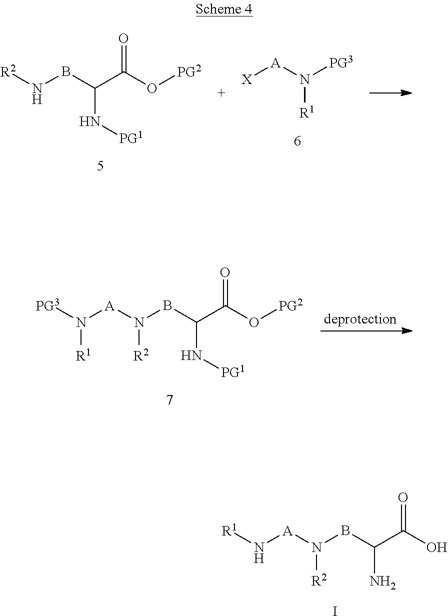

According to Scheme 4, a compound of the formula (5), a known compound or a compound prepared by known means wherein PG¹ is a protecting group selected, for example, from the group consisting of triphenylmethyl (trityl), tert-butyloxycarbonyl (BOC), 9-fluorenylmethyloxycarbonyl (FMOC), and carbobenzyloxy (Cbz), and PG² is selected, for example, from the group consisting of 9-fluorenylmethyl (Fm), C1-6 alkyl and C3-7 branched alkyl, is reacted with a compound of the formula (6), a known compound or compound prepared by known methods wherein X is a leaving group such as bromine, chlorine, iodine, methanesulfonate, tolylsulfonate, and the like, and PG³ is a protecting group selected from the group consisting of, for example, tert-butyloxycarbonyl (BOC), 9-fluorenylmethyloxycarbonyl (FMOC), and carbobenzyloxy (Cbz), in the presence of a base such as trimethylamine, diisopropylethylamine, pyridine, potassium carbonate, cesium carbonate, sodium hydroxide, potassium hydroxide, sodium hydride, potassium hydride, lithium diisopropylamide, sodium hexamethyldisilazide, lithium hexamethyldisilazide, potassium t-butoxide, sodium t-butoxide, and the like, in a solvent such as tetrahydrofuran, 1,4-dioxane, methylene chloride, methanol, ethanol, t-butanol, and the like, optionally with heating, optionally with microwave irradiation to provide a compound of the formula (7).

According to Scheme 4, a compound of the formula (7) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (7) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I).

Alternatively, according to Scheme 4, a compound of the formula (7) is then reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (I).

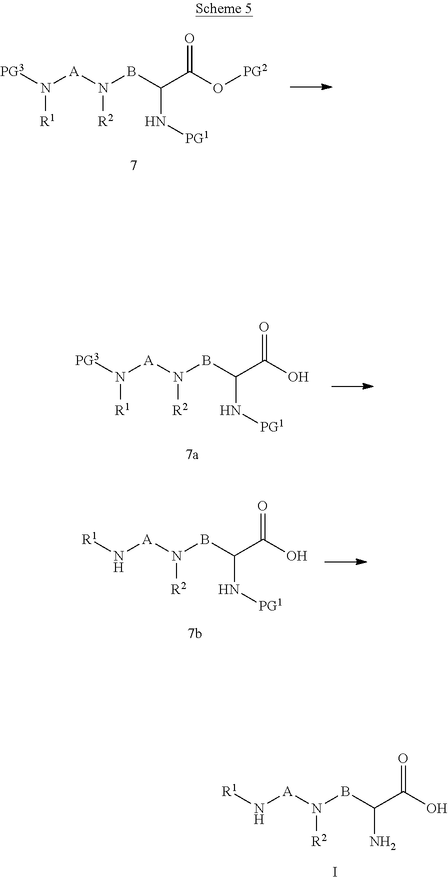

According to Scheme 5, a compound of the formula (7) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (7a). Alternatively, a compound of the formula (7) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (7a).

According to Scheme 5, a compound of the formula (7a) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (7b). Alternatively, a compound of the formula (7a) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (7b). Alternatively, a compound of the formula (7a) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (7b). A compound of the formula (7b) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (7b) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (I). Alternatively, a compound of the formula (7b) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

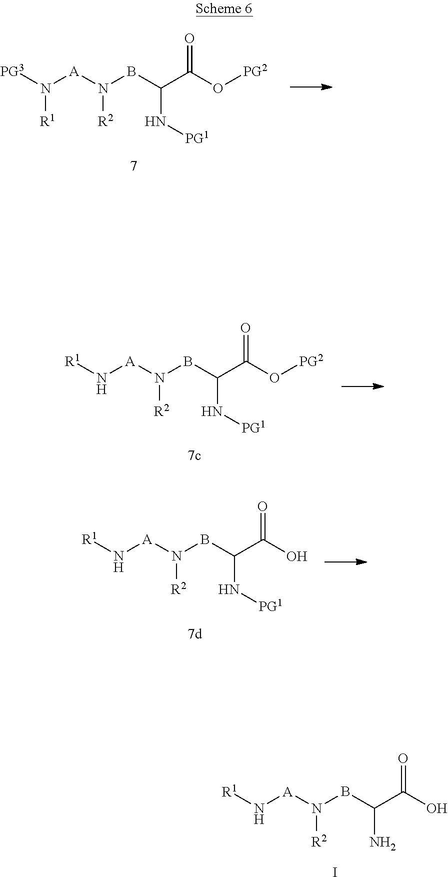

According to Scheme 6, a compound of the formula (7) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (7c). Alternatively, a compound of the formula (7) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (7c). Alternatively, a compound of the formula (7) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (7c).

According to Scheme 6, a compound of the formula (7c) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (7d). Alternatively, a compound of the formula (7c) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (7d).

According to Scheme 6, a compound of the formula (7d) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (7d) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (I). Alternatively, a compound of the formula (7d) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

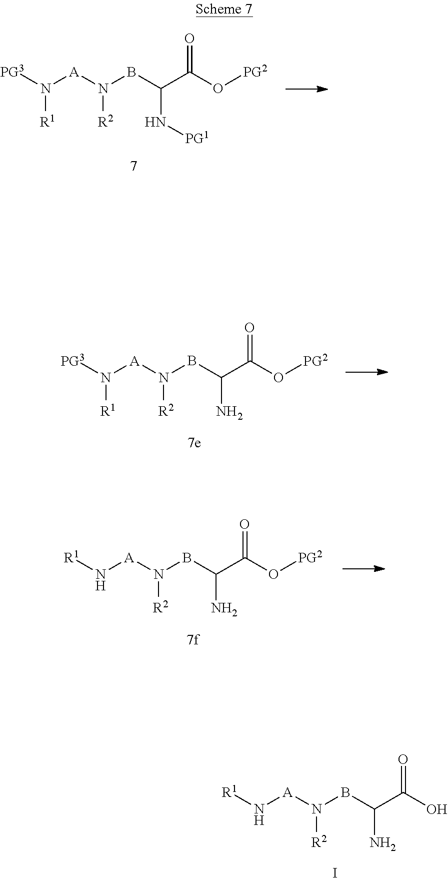

According to Scheme 7, a compound of the formula (7) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (7e). Alternatively, a compound of the formula (7) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (7e). Alternatively, a compound of the formula (7) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (7e).

According to Scheme 7, a compound of the formula (7e) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (7f). Alternatively, a compound of the formula (7e) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (7f).

According to Scheme 7, a compound of the formula (7) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (7f) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofaran, 1,4-dioxane, ethanol, methanol, and the like, optionally with heating, optionally with microwave irradiation to provide a compound of the formula (I). Alternatively, a compound of the formula (7f) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I).

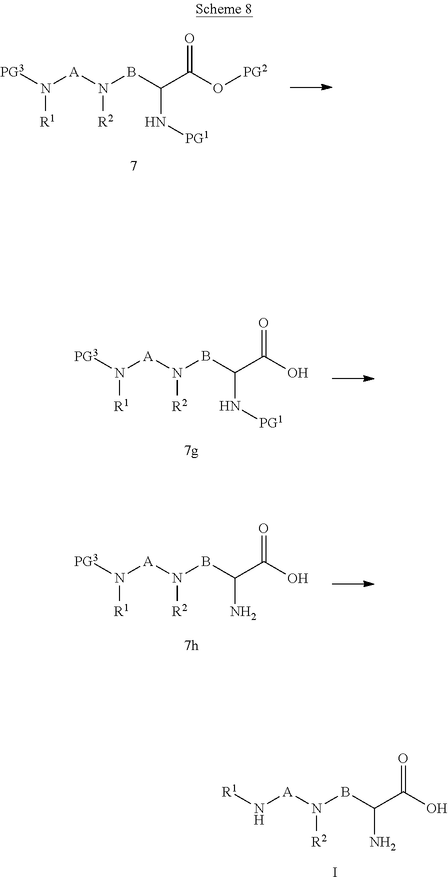

According to Scheme 8, a compound of the formula (7) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (7g). Alternatively, a compound of the formula (7) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (7g).

According to Scheme 8, a compound of the formula (7g) is then reacted with a base such as piperidine, pyridine, 2,6-lutidine, and the like, in a solvent such as methylene chloride, tetrahydrofuran. 1,4-dioxane, ethanol, methanol, and the like, optionally with heating, optionally with microwave irradiation to provide a compound of the formula (7h). Alternatively, a compound of the formula (7g) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (7h). Alternatively, a compound of the formula (7g) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (7h).

According to Scheme 8, a compound of the formula (7h) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (7h) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as, tetrahydrofuran, 1,4-dioxane, ethanol, methanol, and the like, optionally with heating, optionally with microwave irradiation to provide a compound of the formula (I). Alternatively, a compound of the formula (7h) is reacted with an acid such as acetic acid, trifluoroacetic acid, hydrochloric acid, sulfuric acid, and the like, optionally in a solvent such as methylene chloride, tetrahydrofuran, 1,4-dioxane, ethanol, methanol, and the like, optionally with heating, optionally with microwave irradiation to provide a compound of the formula (I).

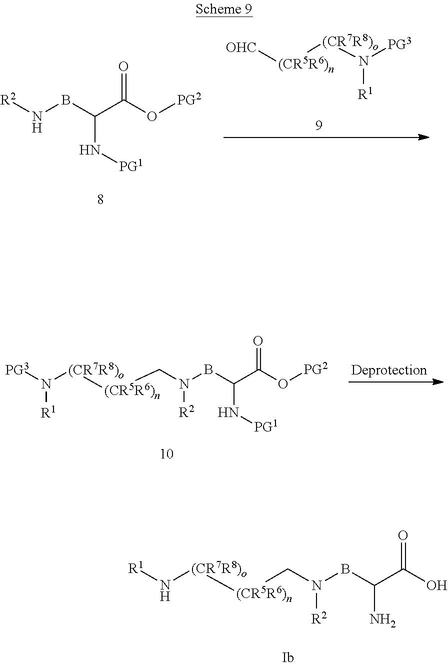

According to Scheme 9, a compound of the formula (9), a known compound or a compound prepared by known means wherein PG¹ is a protecting group selected from the group consisting of, for example, triphenylmethyl (trityl), tert-butyloxycarbonyl (BOC), 9-fluorenylmethyloxycarbonyl (FMOC), and carbobenzyloxy (Cbz) and PG² is selected from the group consisting of, for example, 9-fluorenylmethyl (Fm), C1-6 alkyl and C3-7 branched alkyl, is reacted with a compound of the formula (9), a known compound or a compound prepared by known methods wherein PG³ is a protecting group selected from the group consisting of, for example, triphenylmethyl (trityl), tert-butyloxycarbonyl (BOC), 9-fluorenylmethyloxycarbonyl (FMOC), and carbobenzyloxy (Cbz), in the presence of a reducing agent such as sodium borohydride, lithium borohydride, sodium borohydride, sodium cyanoborohydride, sodium triacetoxy borohydride, lithium triacetoxy borohydride, and the like, optionally in the presence an acid such as acetic acid, formic acid, trifluoroacetic acid, hydrochloric acid, and the like, optionally in the presence of a Lewis acid such as boron trifluoride, aluminum trichloride, titanium tetrachloride, tin chloride, and the like, in the presence of a solvent such as methanol, ethanol, isopropanol, tetrahydrofuran, 1,4-dioxane, methylene chloride, and the like, optionally with heating, optionally with microwave irradiation to provide a compound of the formula (10).

According to Scheme 9, a compound of the formula (8) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (I). Alternatively, a compound of the formula (10) is reacted with a base such as lithium hydroxide, sodium hydroxide, sodium carbonate, lithium carbonate, piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (1). Alternatively, a compound of the formula (10) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (Ib).

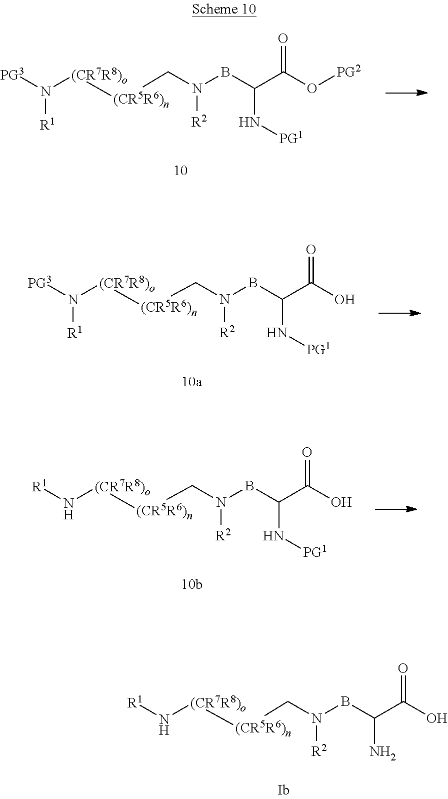

According to Scheme 10, a compound of the formula (10) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (10a). Alternatively, a compound of the formula (10) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (10a).

According to Scheme 10, a compound of the formula (10a) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (10b). Alternatively, a compound of the formula (10a) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (10b). Alternatively, a compound of the formula (10a) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (10b).

According to Scheme 10, a compound of the formula (10b) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (1). Alternatively, a compound of the formula (10b) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (Ib). Alternatively, a compound of the formula (10b) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (Ib).

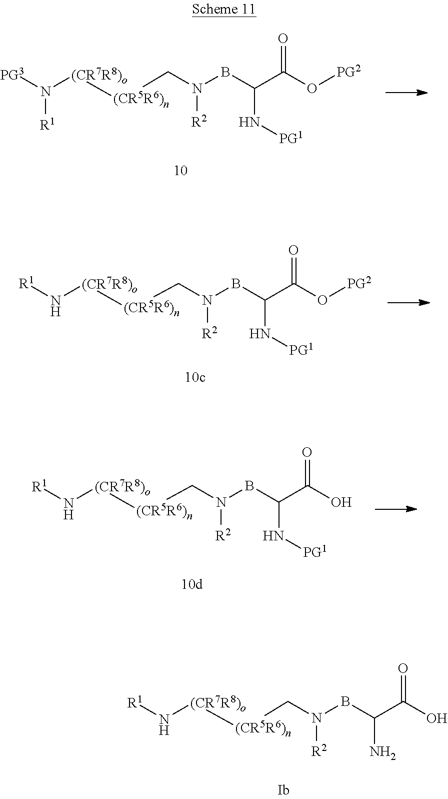

According to Scheme 11, a compound of the formula (10) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (10c). Alternatively, a compound of the formula (10) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (10c). Alternatively, a compound of the formula (10) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (10c).

According to Scheme 11, a compound of the formula (10c) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (10d). Alternatively, a compound of the formula (10c) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (10d).

A compound of the formula (10d) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (Ib). Alternatively, a compound of the formula (10d) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (Ib). Alternatively, a compound of the formula (10d) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (Ib).

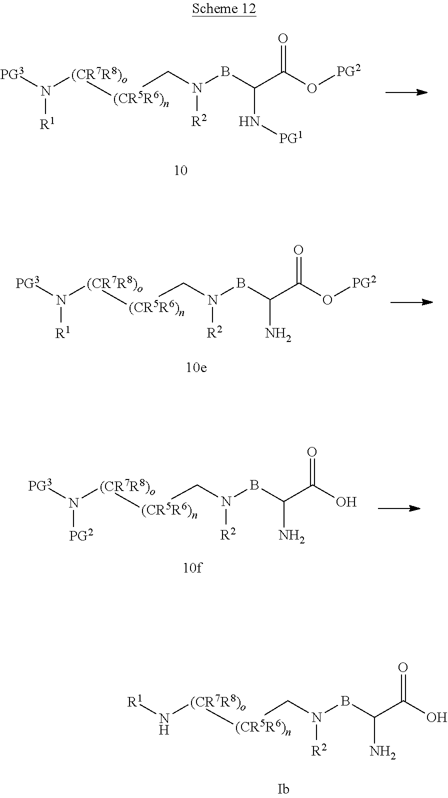

According to Scheme 12, a compound of the formula (10) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (10e). Alternatively, a compound of the formula (10) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (10e). Alternatively, a compound of the formula (10) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (10e).

According to Scheme 12, a compound of the formula (10e) is then reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (10f). Alternatively, a compound of the formula (10e) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (10f).

According to Scheme 12, a compound of the formula (Of) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (Ib). Alternatively, a compound of the formula (10f) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (Ib). Alternatively, a compound of the formula (10f) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (Ib).

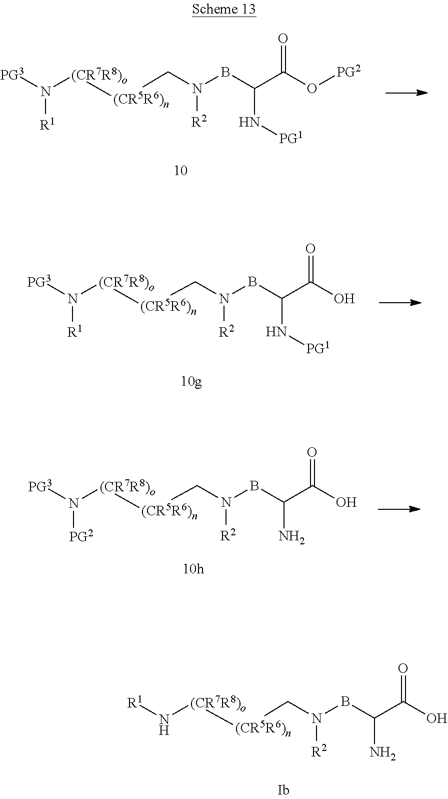

According to Scheme 13, a compound of the formula (10) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (10g). Alternatively, a compound of the formula (10) is reacted with a base such as lithium hydroxide, sodium hydroxide, sodium carbonate, lithium carbonate, and the like in a solvent such as methylene chloride, tetrahydrofuran, 1,4-dioxane, ethanol, methanol, and the like, optionally with heating, optionally with microwave irradiation to provide a compound of the formula (10g).

According to Scheme 13, a compound of the formula (10g) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (10h). Alternatively, a compound of the formula (10g) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene] dichloropalladium, and the like, in the presence of a solvent such as tetrahydrofuran, 1,4-dioxane, ethanol, methanol, and the like, optionally with heating, optionally with microwave irradiation to provide a compound of the formula (10h). Alternatively, a compound of the formula (10g) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (10h).

According to Scheme 13, a compound of the formula (10h) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (Ib). Alternatively, a compound of the formula (10h) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene] dichloropalladium, and the like, in the presence of a solvent such as tetrahydrofuran, 1,4-dioxane, ethanol, methanol, and the like, optionally with heating, optionally with microwave irradiation to provide a compound of the formula (b). Alternatively, a compound of the formula (10h) is reacted with an acid such as acetic acid, trifluoroacetic acid, hydrochloric acid, sulfuric acid, and the like, optionally in a solvent such as methylene chloride, tetrahydrofuran, 1,4-dioxane, ethanol, methanol, and the like, optionally with heating, optionally with microwave irradiation to provide a compound of the formula (Ib).

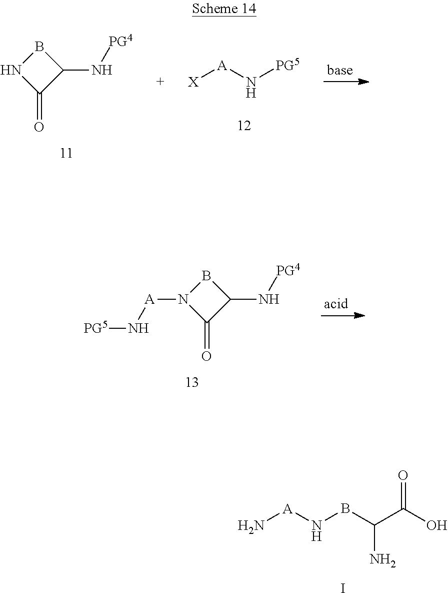

According to Scheme 14, a compound of the formula (11), a known compound or a compound prepared by known methods wherein PG⁴ is a protecting group selected from the group consisting of, for example, triphenylmethyl (trityl), tert-butyloxycarbonyl (BOC), 9-fluorenylmethyloxycarbonyl (FMOC), and carbobenzyloxy (Cbz) is reacted with a compound of the formula (12), a known compound or a compound prepared by known methods wherein X is a leaving group such as bromine, chlorine, iodine, methanesulfonate, tolylsulfonate, and the like and PG⁵ is a protecting group selected from the group consisting of, for example, triphenylmethyl (trityl), tert-butyloxycarbonyl (BOC), 9-fluorenylmethyloxycarbonyl (FMOC), and carbobenzyloxy (Cbz) in the presence of a base such as sodium hydride, potassium hydride, lithium diisopropylamide, sodium hexamethyldisilazide, lithium hexamethyldisilazide, potassium t-butoxide, sodium t-butoxide and the like, in a solvent such as tetrahydrofuran, 1,4-dioxane, methylene chloride, methanol, ethanol, t-butanol, and the like, optionally with heating, optionally with microwave irradiation to provide a compound of the formula (13).

According to Scheme 14, a compound of the formula (13) is then reacted with an acid such as hydrochloric acid, sulfuric acid, phosphoric acid and the like, optionally in the presence of water, optionally with heating, optionally with microwave irradiation to provide a compound of formula I.

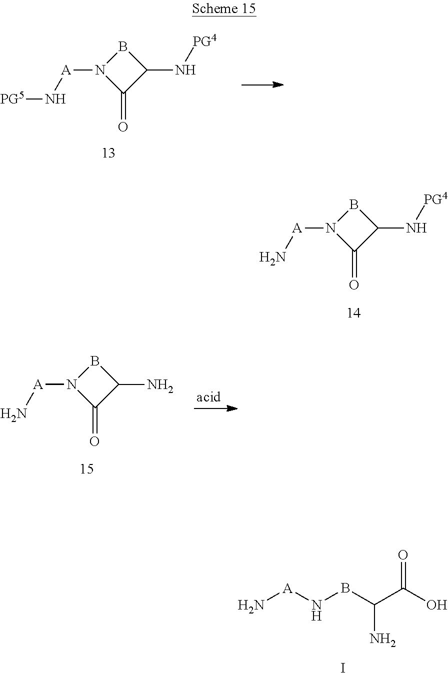

According to Scheme 15, compound of the formula (13) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (14). Alternatively, a compound of the formula (13) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (14). Alternatively, a compound of the formula (13) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (14).

According to Scheme 15, a compound of the formula (14) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (15). Alternatively, a compound of the formula (14) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (15). Alternatively, a compound of the formula (14) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (15).

According to Scheme 15, a compound of the formula (15) is then reacted with an acid such as hydrochloric acid, sulfuric acid, phosphoric acid and the like, optionally in the presence of water, optionally with heating, optionally with microwave irradiation to provide a compound of formula I.

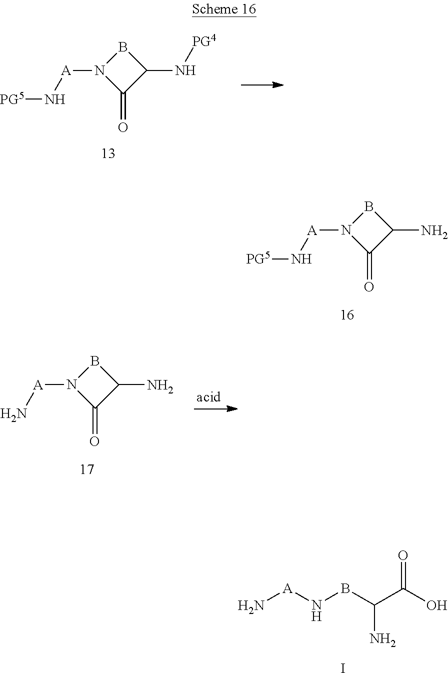

According to Scheme 16, a compound of the formula (13) is reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (16). Alternatively, a compound of the formula (13) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (16). Alternatively, a compound of the formula (13) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (16).

According to Scheme 16, a compound of the formula (16) is then reacted with a base such as piperidine, pyridine, 2,6-lutidine, and the like, in a solvent such as methylene chloride, tetrahydrofuran, 1,4-dioxane, ethanol, methanol, and the like, optionally with heating, optionally with microwave irradiation to provide a compound of the formula (17). Alternatively, a compound of the formula (16) is reacted with hydrogen in the presence of a catalyst such as palladium on carbon, palladium on barium sulfate, palladium on celite, palladium on calcium carbonate, palladium on barium carbonate, palladium on silica, palladium on alumina, palladium acetate, palladium bis(triphenylphosphine) dichloride, palladium tetrakis(triphenylphospine), bis(acetonitrile) dichloropalladium [1,1′-bis(diphenylphosphino) ferrocene]dichloropalladium, and the like, in the presence of a solvent such as tetrahydrofuran, 1,4-dioxane, ethanol, methanol, and the like, optionally with heating, optionally with microwave irradiation to provide a compound of the formula (17). Alternatively, a compound of the formula (16) is reacted with an acid such as acetic acid, trifluoroacetic acid, hydrochloric acid, sulfuric acid, trifluoromethanesulfonic acid and the like, optionally in a solvent such as methylene chloride, tetrahydrofuran, 1,4-dioxane, ethanol, methanol, and the like, optionally with heating, optionally with microwave irradiation to provide a compound of the formula (17).

According to Scheme 16, a compound of the formula (17) is then reacted with an acid such as hydrochloric acid, sulfuric acid, phosphoric acid and the like, optionally in the presence of water, optionally with heating, optionally with microwave irradiation to provide a compound of formula (I).

In the aforesaid processes, certain functional groups which would be sensitive to the reaction conditions may be protected by protecting groups. A protecting group is a derivative of a chemical functional group which would otherwise be incompatible with the conditions required to perform a particular reaction which, after the reaction has been carried out, can be removed to re-generate the original functional group, which is thereby considered to have been “protected”. Any chemical functionality that is a structural component of any of the reagents used to synthesize compounds of this invention may be optionally protected with a chemical protecting group if such a protecting group is useful in the synthesis of compounds of this invention. The person skilled in the art knows when protecting groups are indicated, how to select such groups, and processes that can be used for selectively introducing and selectively removing them, because methods of selecting and using protecting groups have been extensively documented in the chemical literature. Techniques for selecting, incorporating and removing chemical protecting groups may be found, for example, in Protective Groups in Organic Synthesis by Theodora W. Greene, Peter G. M. Wuts, John Wiley & Sons Ltd., the entire disclosure of which is incorporated herein by reference.

It will be appreciated by one skilled in the art that the processes described are not the exclusive means by which compounds of Formula I may be synthesized and that a repertoire of synthetic organic reactions is available to be potentially employed in synthesizing compounds of the invention. The person skilled in the art knows how to select and implement appropriate synthetic routes. Suitable synthetic methods may be identified by reference to the literature, including reference sources such as Comprehensive Organic Synthesis, Ed. B. M. Trost and I. Fleming (Pergamon Press, 1991), Comprehensive Organic Functional Group Transformations, Ed. A. R. Katritzky, O. Meth-Cohn, and C. W. Rees (Pergamon Press, 19%), Comprehensive Organic Functional Group Transformations II, Ed. A. R. Katritzky and R. J. K. Taylor (Editor) (Elsevier, 2^{nd} Edition, 2004), Comprehensive Heterocyclic Chemistry, Ed. A. R. Katritzky and C. W. Rees (Pergamon Press, 1984), and Comprehensive Heterocyclic Chemistry II, Ed. A. R. Katritzky, C. W. Rees, and E. F. V. Scriven (Pergamon Press, 1996).

The compounds of Formula I and intermediates may be isolated from their reaction mixtures and purified by standard techniques such as filtration, liquid-liquid extraction, solid phase extraction, distillation, recrystallization or chromatography.

It will be understood that when compounds of Formula I the present invention contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention which are biologically active in the treatment of insulin resistance.

A chiral center occurs in the α-carbon of the α-amino acid functionality of the compounds of Formula I. The compounds of Formula I are characterized by the (S) absolute configuration about the α-carbon of the contained α-amino acid functionality, according to the Cahn-Ingold-Prelog rules, as exampled by the compound (S)-2-amino-6-((6-aminohexyl)amino)hexanoic acid, a compound of Formula I:

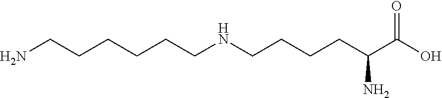 [SMILES: NCCCCCCNCCCC[C@H](N)C(=O)O]

According to certain embodiments, a compound of Formula I is an isolated (S) optical isomer with respect to the configuration about the α-carbon of the contained α-amino acid functionality. By an “isolated optical isomer” means a compound which has been substantially purified from the corresponding optical isomer(s) of the same formula. Preferably, the isolated isomer is at least about 80%, more preferably at least 85% pure, more preferably at least 90% pure, more preferably at least 95% pure, even more preferably at least 98% pure, most preferably at least about 99% pure, by weight, the balance being made up of the corresponding (R) enantiomer. In some embodiments, the isolated (S) enantiomer is free of the corresponding (R) enantiomer, except for trance amounts of the (R) enantiomer.

Methods of Preventing and/or Treating Pre-Diabetes and Type 2 Diabetes

Embodiments of the invention are directed to methods of preventing or treating type 2 diabetes in a subject in need thereof comprises administering to the subject a therapeutically effective amount of a compound of formula I-III as described herein.

Embodiments of the invention are directed to methods of preventing or treating pre-diabetes in a subject in need thereof comprises administering to the subject a therapeutically effective amount of a compound of formula I-III as described herein.

Embodiments of the invention are directed to methods of preventing or treating a condition in a subject in need thereof comprises administering to the subject a therapeutically effective amount of a compound of formula I-III as described herein, wherein the condition is characterized by an increase in a measurement selected from the group consisting of AiC, glucose, insulin, homeostasis model of assessment of insulin resistance (HOMA-IR), oxidative stress in adipose tissue, and carbonylation of GLUT4.

Type 2 diabetes is a disease diagnosed by a set of standard characteristics known in the art. Pre-diabetes is similarly diagnosed based upon a set of standard characteristics known in the art.

Conditions characterized by an increase in the levels of A1C, glucose, insulin, homeostasis model of assessment of insulin resistance (HOMA-IR), oxidative stress in adipose tissue, and/or carbonylation of GLUT4 lead to a diagnosis of insulin resistance, pre-diabetes, or type 2 diabetes. Subjects with a condition as described herein are at higher risk of developing pre-diabetes, type 2 diabetes, hyperglycemia, dyslipidemia, hypertension, artherosclerotic cardiovascular disease (ASCVD), cardiometabolic disease, chronic kidney disease, early nephropathy, retinopathy, cardiovascular disease and biomechanical complications. In embodiments, the subject is treated by the administration of the compound of formula I-III wherein the symptoms of the condition is treated.

In embodiments, the therapeutically effective amount of the compound of formula I-III is between about 300 mg to about 500 mg. In embodiments, the therapeutically effective amount of the compound of formula I-III is between about 500 mg to about 1,000 mg. In embodiments, the therapeutically effective amount of the compound of formula I-III for preventing or treating pre-diabetes is between about 300 mg to about 500 mg. In embodiments, the therapeutically effective amount of the compound of formula I-III for treating type 2 diabetes is between about 500 mg to about 1000 mg. In embodiments, the treating or preventing involves administering of the compound of formula I-III and one or more additional therapeutic agents.

In embodiments, the preventing or treating type 2 diabetes may require breakthrough therapy, wherein breakthrough therapy comprises the administration of one or more additional therapeutic agents. In embodiments, the preventing or treating type 2 diabetes in a subject in need thereof comprises administering to the subject a therapeutically effective amount of a compound of formula I-III, wherein therapeutically effective amount of a compound of formula I-III is between about 300 mg to about 500 mg.

Treatment efficacy is generally determined by improvement in insulin resistance, i.e., an increase in insulin sensitivity. Insulin resistance and beta-cell function may be assessed before, during, and after treatment by use of the homeostasis model assessment of insulin resistance (HOMA-IR) index. The HOMA-IR value is calculated as the level of fasting glucose (millimoles/liter) times the level of fasting insulin (microunits/milliliter) divided by 22.5. The value of 3.0 identifies the highest quartile among populations without diabetes. In embodiments, administration of a compound of formula I-III prevents the HOMA-R value from increasing above 3. In embodiments, administration of a compound of formula I-III treats the subject in need thereof, wherein the HOMA-IR value is decreased.

Treatment efficacy may be assessed by the A I C test, which is the average of an individual's blood glucose level over the prior 3 months. The A1C test is based on the attachment of glucose to hemoglobin. In the body, red blood cells are constantly forming and dying, but typically they live for about 3 months. Thus, the AiC test reflects the average of a person's blood glucose levels over the past 3 months. The A1C test result is reported as a percentage. The higher the percentage, the higher a person's blood glucose levels have been. A normal A1C level is below 5.7%, pre-diabetes A1C level is a range of between 5.7% to 6.4%, and type 2 diabetes A1C level is equal to or greater than 6.5%. In embodiments, administration of a compound of formula I-III to a subject in need thereof decreases the A1C level to less than 6.5% or less than 5.7%.

Treatment efficacy may be assessed by measuring the level of glucose in the blood. This is done, for instance, using a fasting blood glucose) test, and/or a glucose tolerance test.

Treatment efficacy may be assessed by measuring the level of insulin in the blood. A normal fasting insulin level is below 5. A fasting insulin level around 8.0 results in twice the risk of pre-diabetes, and a fasting insulin of about 25 results in about a five times the risk of prediabetes.

Treatment efficacy may be assessed by measuring the level of oxidative stress in adipose tissue.

Treatment efficacy may be assessed by measuring the level of carbonylation of GLUT4. In adipose tissue during overnutrition, oxidative stress results in extensive oxidation and carbonylation of numerous proteins, including carbonylation of GLUT4 near the glucose transport channel, which results in the loss of GLUT4 activity. The carbonylation and oxidation-induced inactivation of GLUT4 may result in insulin resistance. In embodiments, administration of a compound of formula I-III to a subject in need thereof decreases the level of GLUT4 carbonylation.

### Pharmaceutical Compositions

A pharmaceutical composition comprises a pharmaceutically acceptable carrier and a compound, or a pharmaceutically acceptable salt thereof, according to Formula I-III.

The compounds may be administered in the form of a pharmaceutical composition, in combination with a pharmaceutically acceptable carrier. The compound of Formula I-III may comprise from about 0.1 to about 99.99 weight percent of the formulation.

The compositions are preferably formulated in a unit dosage form, each dosage containing from about 1 to about 1,000 mg, more typically from about 1 to about 500 mg, more typically from about 10 to about 100 mg, per unit dosage.

The compound of Formula I-III is preferably administered with a pharmaceutically acceptable carrier selected on the basis of the selected route of administration and standard pharmaceutical practice. The compound of Formula I-III may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa. Suitable dosage forms may comprise, for example, tablets, capsules, solutions, parenteral solutions, troches, suppositories, or suspensions.

For parenteral administration, the compound of Formula I-III may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or non-aqueous solution, dispersion, suspension or emulsion.

For oral administration, the compound of Formula I-III may be combined with one or more solid inactive ingredients for the preparation of tablets, capsules, pills, powders, granules or other suitable oral dosage forms. For example, the compound of Formula I-III may be combined with at least one excipient such as fillers, binders, humectants, disintegrating agents, solution retarders, absorption accelerators, wetting agents, absorbents, or lubricating agents. According to one tablet embodiment, the compound of Formula I-III may be combined with carboxymethylcellulose calcium, magnesium stearate, mannitol and starch, and then formed into tablets by conventional tableting methods.

The pharmaceutical compositions of the present invention may also be formulated so as to provide slow or controlled release of the compound of Formula I-III therein using, for example but not limited to, hydropropylmethyl cellulose in varying proportions to provide the desired release profile, other polymer matrices, gels, permeable membranes, osmotic systems, multilayer coatings, microparticles, liposomes and/or microspheres.

In general, a controlled-release preparation is a pharmaceutical composition capable of releasing the compound of Formula I-III at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations.

The controlled-release of the compound of Formula I-III may be stimulated by various inducers, for example pH, temperature, enzymes, water, or other physiological conditions or compounds. Various mechanisms of drug release exist. For example, in one embodiment, the controlled-release component may swell and form porous openings large enough to release the compound of Formula I-III after administration to a patient. The term “controlled-release component” in the context of the present invention is defined herein as a compound or compounds, such as polymers, polymer matrices, gels, permeable membranes, liposomes and/or microspheres, that facilitate the controlled-release of the compound of Formula I-III in the pharmaceutical composition. In another embodiment, the controlled-release component is biodegradable, induced by exposure to the aqueous environment, pH, temperature, or enzymes in the body. In another embodiment, sol-gels may be used, wherein the compound of Formula I-III is incorporated into a sol-gel matrix that is a solid at room temperature. This matrix is implanted into a patient, preferably a mammal, having a body temperature high enough to induce gel formation of the sol-gel matrix, thereby releasing the compound of Formula I-III into the patient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

In embodiments, the compound of Formula I-III is in a pharmaceutical composition. In embodiments, a pharmaceutical composition of the disclosure comprises a carrier and/or diluent appropriate for its delivering by injection to a human or animal organism. Such carrier and/or diluent is non-toxic at the dosage and concentration employed. It is selected from those usually employed to formulate compositions for parental administration in either unit dosage or multi-dose form or for direct infusion by continuous or periodic fusion.

A physician will determine the dosage of the compound of Formula I-III which will be most suitable and it will vary with the form of administration and the particular compound chosen, and furthermore, it will vary depending upon various factors, including but not limited to the patient under treatment, the age of the patient, the weight of the patient, the severity of the condition being treated, the route of administration, and the like. It will generally be found that when the composition is administered orally, larger quantities of the compound of Formula I-III will be required to produce the same effect as a smaller quantity given parenterally. The compounds of Formula I-III may be administered in a convenient manner. Suitable topical routes include oral, rectal, inhaled (including nasal), topical (including buccal and sublingual), transdermal and vaginal, preferably across the epidermis. The compound of Formula I-III can also be used for parenteral administration (including subcutaneous, intravenous, intramuscular, intradermal, intraarterial, intrathecal and epidural), and the like. It will be appreciated that the preferred route may vary with, for example, the condition of the recipient. In embodiments, the therapeutically effective amount of a compound of Formula I-III may be from about 10 mg/day to about 2,000 mg/day. In embodiments, the therapeutically effective amount of a compound of Formula I-III may be from about 10 mg/day to about 1,000 mg/day. In embodiments, the therapeutically effective amount of a compound of Formula I-III may be from about 10 mg/day to about 500 mg/day. In embodiments, the therapeutically effective amount of a compound of Formula I-III may be from about 10 mg/day to about 100 mg/day.

For example, a daily dosage from about 0.05 to about 50 mg/kg/day may be utilized, more preferably from about 0.1 to about 10 mg/kg/day. Higher or lower doses are also contemplated as it may be necessary to use dosages outside these ranges in some cases. The daily dosage may be divided, such as being divided equally into two to four times per day daily dosing.

The treatment may be carried out for as long a period as necessary, either in a single, uninterrupted session, or in discrete sessions. The treating physician will know how to increase, decrease, or interrupt treatment based on patient response. The treatment schedule may be repeated as required. According to one embodiment, compound of Formula I-III is administered once daily.

In embodiments, the methods may include the co-administration of one or more additional therapeutic agents. In embodiments, co-administration may be part of the same pharmaceutical composition or separate pharmaceutical compositions described herein. In embodiments, co-administration may be at the same time, substantially the same time, before or after administration of the compositions described herein.

The practice of the disclosed subject matter is illustrated by the following non-limiting examples.

### EXAMPLES

### Example 1: (S)-2-amino-6-((6-aminohexyl)amino)hexanoic acid trihydrochloride

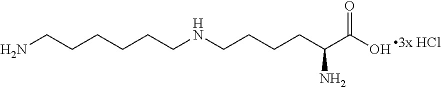 [SMILES: NCCCCCCNCCCC[C@H](N)C(=O)O]

### Preparation of tert-butyl (S)-(1-(6-((tert-butoxycarbonyl)amino)hexyl)-2-oxoazepan-3-yl)carbamate

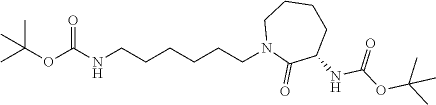 [SMILES: CC(C)(C)OC(=O)NCCCCCCN1CCCC[C@H](NC(=O)OC(C)(C)C)C1=O]

Lithium bis(trimethylsilyl)amide (0.8760 mmol; 876 μL of a 1.0 M solution in THF) was added to a solution of tert-butyl (S)-(2-oxoazepan-3-yl)carbamate (0.4380 mmol; 100 mg) in anhydrous tetrahydrofuran (1 mL). The resulting suspension was stirred at room temperature for thirty minutes. N-Boc-6-bromohexylamine (0.8760 mmol; 245 mg) was added all at once. The reaction was stirred at room temperature for 48 hours and then at 60° C. for 18 hours. It was concentrated down and the residue was partitioned between ethyl acetate and water. The aqueous layer was removed. The organic layer was washed with brine, dried over anhydrous sodium sulfate and concentrated. The crude product was purified by column chromatography on silica gel using a gradient solvent system of 0 to 40% of ethyl acetate in hexanes to afford the titled compound as a colorless oil. ¹H NMR (400 MHz, CDCl₃) δ 6.00 (bd, J=5.8 Hz, 1H), 4.56 (bs, 1H), 4.33 (m, 1H), 3.43-3.52 (m, 2H), 3.36-3.41 (m, 1H), 3.25-3.33 (m, 1H), 3.15-3.23 (m, 1H), 3.05-3.13 (m, 2H), 1.89-1.98 (m, 1H), 1.74-1.88 (m, 3H), 1.41-1.54 (m, 22H), 1.27-1.36 (m, 5H); MS (ESI): m/z 428.3 [(M+H)⁺].

### Preparation of (S)-2-amino-6-((6-aminohexyl)amino)hexanoic acid trihydrochloride

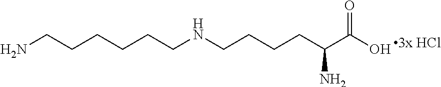 [SMILES: NCCCCCCNCCCC[C@H](N)C(=O)O]

tert-Butyl-(S)-(1-(6-((tert-butoxycarbonyl)amino)hexyl)-2-oxoazepan-3-yl)carbamate (0.0215 mmol; 9.2 mg) was dissolved into 12 N aqueous hydrochloric acid (1 mL). This solution was stirred at room temperature until all of the bubbling had ceased. It was transferred to a microwave reaction vial and heated at 160° C. for ninety minutes. Upon concentration, pure titled compound was afforded as a pale yellow oil. ¹H NMR (400 MHz, D₂O) δ 4.08 (t, J=6.4 Hz, 1H), 2.98-3.10 (m, 6H), 1.92-2.06 (m, 2H), 1.64-1.81 (m, 6H), 1.40-1.57 (m, 6H); MS (ESI): m/z 246.2 [(M+H)⁺].

### Example 2: (S)-2-amino-5-((6-aminohexyl)amino)pentanoic acid trihydrochloride

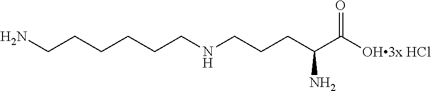

### Preparation of tert-butyl (6-oxohexyl)carbamate

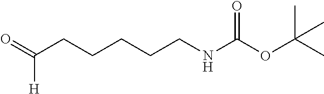

Anhydrous dimethyl sulfoxide (83 uL) was added dropwise to a stirred solution of oxalyl chloride (50 uL) in anhydrous dichloromethane (2 mL) at −78° C. After stirring for 15 minutes, a solution of 6-(tert-butoxy-carbonylamino)-1-hexanol (115 mg, 0.53 mmol) in anhydrous dichloromethane (1 mL) was added dropwise. The resulting mixture was stirred at −78° C. for 45 minutes. Triethylamine (368 uL) was added and the reaction was allowed to warm to room temperature. This solution was concentrated on a rotary evaporator to afford the titled compound as an off-white solid (86 mg, 75% yield) which was used without further purification.

### Preparation of (S)-2-(((benzyloxy)carbonyl)amino)-5-((6-((tert-butoxycarbonyl)amino)hexyl)amino)-pentanoic acid

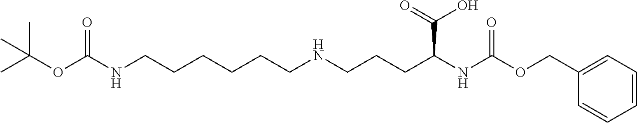 [SMILES: CC(C)(C)OC(=O)NCCCCCCNCCC[C@H](NC(=O)OCc1ccccc1)C(=O)O]

To a stirred suspension of N-alpha-benzyloxycarbonyl-L-ornithine 94 mg, (0.352 mmol) in anhydrous methanol (2 mL) containing acetic acid (100 uL) was added a solution of tert-butyl (6-oxohexyl)carbamate (114 mg, 0.528 mmol) in anhydrous methanol (1.9 mL). The resulting mixture was stirred at room temperature for 30 minutes. Sodium cyanoborohydride (66 mg, 1.057 mmol) was then added and the reaction was stirred at room temperature overnight. After concentration on a rotary evaporator, the residue was partitioned between ethyl acetate and IM aqueous potassium bisulfate. The aqueous layer was removed. The organic phase was washed with water and brine, dried over anhydrous sodium sulfate and concentrated on a rotary evaporator. The resulting residue was purified by reversed phase chromatography (C18 column) using a gradient of 10 to 100% acetonitrile in water with 0.1% formic acid modifier. The titled compound (87 mg, 53% yield) was obtained as a pale yellow oil. ¹H NMR (400 MHz, D₂O) δ 3.94 (t, J=5.92 Hz, 0.5H), 3.63 (m, 0.5H), 2.99-3.13 (m, 6H), 1.65-2.04 (m, 8H), 1.43 (m, 4H); MS (ESI): m/z 466.2 [(M+H)⁺].

### Preparation of (S)-2-Amino-5-((6-aminohexyl)amino)pentanoic acid trihydrochloride

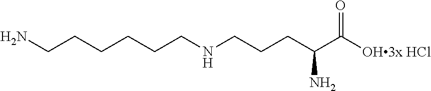

A solution of (S)-2-(((benzyloxy)carbonyl)amino)-5-((6-((tert-butoxycarbonyl)amino)hexyl)amino)pentanoic acid (18 mg, 0.039 mmol)) in 6N aqueous hydrochloric acid (4 mL) was refluxed for two hours. This solution was concentrated on a rotary evaporator to afford the titled compound (12 mg, 90% yield) as a pale yellow oil. ¹H NMR (400 MHz, D₂O) δ 4.27 (m, 0.5H), 3.95 (m, 0.5H), 3.33-3.48 (m, 6H), 2.00-2.37 (m, 811), 1.77 (m, 4H); MS (ESI): m/z 232.2 [(M+H)⁺].

### Example 3: (S)-2-amino-5-((5-aminopentyl)amino)pentanoic acid trihydrochloride

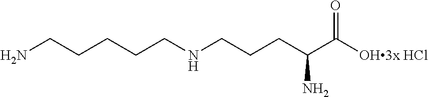

### Preparation of tert-butyl (5-oxopentyl)carbamate

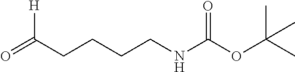

Anhydrous dimethyl sulfoxide (58 uL) was added dropwise to a stirred solution of oxalyl chloride (35 uL) in anhydrous dichloromethane (1.5 mL) at −78° C. After stirring for 15 minutes, a solution of 6-(tert-butoxy-carbonylamino)-1-pentanol (75 mg, 0.37 mmol) in anhydrous dichloromethane (0.75 mL) was added dropwise. The resulting mixture was stirred at −78° C. for 45 minutes. Triethylamine (257 uL) was added and the reaction was allowed to warm to room temperature. This solution was concentrated on a rotary evaporator to afford the titled compound as an off-white solid (52 mg, 70% yield) which was used without further purification.

### Preparation of (S)-2-(((benzyloxy)carbonyl)amino)-5-((5-((tert-butoxycarbonyl)amino)pentyl)amino)-pentanoic acid

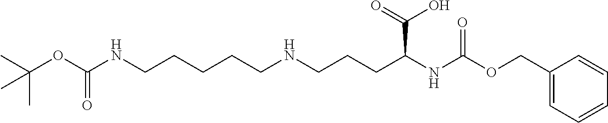 [SMILES: CC(C)(C)OC(=O)NCCCCCNCCC[C@H](NC(=O)OCc1ccccc1)C(=O)O]

To a stirred suspension of N-alpha-benzyloxycarbonyl-L-ornithine 35 mg, (0.13 mmol) in anhydrous methanol (1 mL) containing acetic acid (38 uL) was added a solution of tert-butyl (5-oxopentyl)carbamate (40 mg, 0.20 mmol) in anhydrous methanol (1.0 mL). The resulting mixture was stirred at room temperature for 30 minutes. Sodium cyanoborohydride (25 mg, 0.40 mmol) was then added and the reaction was stirred at room temperature overnight. After concentration on a rotary evaporator, the residue was partitioned between ethyl acetate and IM aqueous potassium bisulfate. The aqueous layer was removed. The organic phase was washed with water and brine, dried over anhydrous sodium sulfate and concentrated on a rotary evaporator. The resulting residue was purified by reversed phase chromatography (C18 column) using a gradient of 10 to 100% acetonitrile in water with 0.1% formic acid modifier. The titled compound (34 mg, 58% yield) was obtained as a colorless oil. ¹H NMR (400 MHz, CD₃OD) δ 7.26-7.38 (m, 5H), 5.08 (s, 2H), 4.03 (m, 1H), 2.90-3.07 (m, 6H), 1.87 (m, 1H), 1.65-1.79 (m, 5H), 1.34-1.54 (m, 13H); MS (ESI): m/z 452.30 [(M+H)⁺].

### Preparation of (S)-2-amino-5-((5-aminopentyl)amino)pentanoic acid trihydrochloride

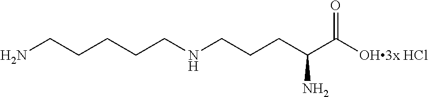

A solution of (S)-2-(((benzyloxy)carbonyl)amino)-5-((5-((tert-butoxycarbonyl) amino)pentyl)amino)pentanoic acid (20 mg, 0.044 mmol)) in 6N aqueous hydrochloric acid (4 mL) was refluxed for two hours. This solution was concentrated on a rotary evaporator to afford the titled compound (12 mg, 88% yield) as a pale yellow oil. ¹H NMR (400 MHz, CD₃OD) δ 4.06 (t, J=5.36 Hz, 1H), 3.06 (m, 4H), 2.96 (t, J=7.52 Hz, 2H), 1.88-2.10 (m, 4H), 1.6-1.83 (m, 4H), 1.47-1.55 (m, 2H); MS (ESI): m/z 218.2 [(M+H)+].

### Example 4: (S)-2-amino-6-(3-aminopropyl)amino)hexanoic acid dihydrochloride

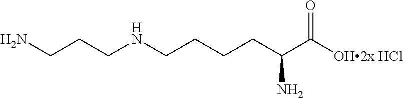

### Preparation of tert-butyl (S)-(2-oxoazepan-3-yl)carbamate

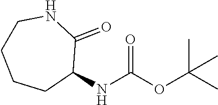 [SMILES: CC(C)(C)OC(=O)N[C@H]1CCCCNC1=O]

Di-tert-butyl-dicarbonate (733 μL, 3.189 mmol) was added to a suspension of L-(−)-α-amino-ε-caprolactam hydrochloride (500 mg, 3.037 mmol) and triethylamine (847 μL, 6.074 mmol) in anhydrous tetrahydrofuiran (4 mL). The resulting suspension was stirred at room temperature overnight and concentrated down. The residual white solid was partitioned between ethyl acetate and water. The aqueous layer was removed. The organic layer was washed twice with 1N aqueous hydrochloric acid, twice with saturated aqueous sodium bicarbonate, once with brine, dried over anhydrous sodium sulfate and concentrated. Pure titled compound was obtained as a white solid. ¹H NMR (400 MHz, CD₃OD) δ 6.45 (bd, J=5.8 Hz, 1H), 4.18-4.30 (m, 1H), 3.17-3.30 (m, 2H), 1.70-2.03 (m, 4H), 1.48-1.57 (m, 1H), 1.45 (s, 9H), 1.28-1.42 (m, 1H); MS (ESI): m/z 250.8 (M+Na)⁺.

### Preparation of tert-butyl (S)-(1-(3-((tert-butoxycarbonyl)amino)propyl)-2-oxoazepan-3-yl)carbamate

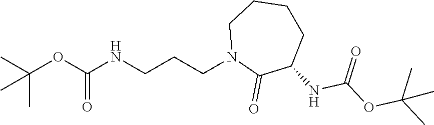 [SMILES: CC(C)(C)OC(=O)NCCCN1CCCC[C@H](NC(=O)OC(C)(C)C)C1=O]

Sodium bis(trimethylsilyl)amide (2.524 mmol; 2.5 mL of a 1.0 M solution in tetrahydrofuran) was added to a solution of tert-butyl (S)-(2-oxoazepan-3-yl)carbamate (288 mg, 1.262 mmol) in anhydrous tetrahydrofuran (12 mL). The resulting suspension was stirred at room temperature for thirty minutes. 3-(Boc-amino)propyl bromide (2.524 mmol; 470 μl) was added all at once and the reaction was stirred at room temperature for 28 hours. The reaction mixture was concentrated on a rotary evaporator and the residue was partitioned between ethyl acetate and water. The aqueous layer was removed. The organic layer was washed with brine, dried over anhydrous sodium sulfate and concentrated. The crude product was purified by column chromatography on silica gel using a gradient solvent system of 0 to 100% of ethyl acetate in hexanes to afford the titled compound as a colorless oil. ¹H NMR (400 MHz, CDCl₃) 5.96 (bd, J=5.0 Hz, 1H), 5.32 (bs, 1H), 4.36 (m, 1H), 3.45-3.62 (m, 2H), 3.33-3.41 (m, 1H), 3.08-3.22 (m, 2H), 2.97-3.06 (m, 1H), 2.02-2.09 (m, 1H), 1.92-2.00 (m, 1H), 1.76-1.87 (m, 2H), 1.61-1.70 (m, 2H), 1.40-1.50 (m, 19H), 1.31-1.38 (m, 1H); MS (ESI): m/z 407.8 (M+Na)⁺.

### Preparation of (S)-2-Amino-6-((3-aminopropyl)amino)hexanoic acid dihydrochloride

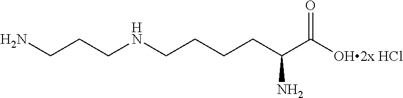

tert-Butyl-(S)-(1-(3-((tert-butoxycarbonyl)amino)propyl)-2-oxoazepan-3-yl)carbamate (100 mg, 0.2596 mmol) was dissolved in 12 N aqueous hydrochloric acid (4 mL). The resulting solution was stirred at room temperature until all of the bubbling had ceased. It was transferred to a microwave reaction vial and heated at 160° C. for ninety minutes. Upon concentration, pure titled compound was afforded as a light yellowish tan solid. ¹H NMR (400 MHz, D₂O) δ 4.00 (t, J=6.3 Hz, 1H), 3.08-3.20 (m, 6H), 1.90-2.15 (m, 4H), 1.72-1.83 (m, 2H), 1.43-1.62 (m, 2H); MS (ESI): m/z 203.9 (M+H)⁺.

### Example 5:GLUT4 Carbonylation

Certain conditions such as overnutrition can lead to oxidative stress and the generation of reactive aldehydes such as 4-HNE. Previous clinical studies have shown that oxidative stress is increased among obese subjects and correlates with the severity of insulin resistance. Of considerable importance, protein carbonyls, as one of the biomarkers of systemic oxidative stress, are also found increased in obesity and type 2 diabetes. Protein carbonyls also positively correlate with the insulin resistance, and significantly decrease after various treatments of obese subjects. Under physiological conditions, intracellular 4-HNE levels are detoxified enzymatically by fatty aldehyde dehydrogenase and glutathione S-transferase A4 (GSTA4). Interestingly, GSTA4 was shown to be decreased in the adipose tissue of obese subjects and in the mouse model of high-fat diet. This seems to indicate that the increased expression of these two aldehyde-oxidizing enzymes could potentially be a good target for reducing protein carbonylations. The studies with GSTA4 null mouse have shown a significant difference in fasting glucose, insulin, and 4-HNE levels as compared to wild-type animals. These studies indicate that the reduction of 4-HNE can be used as a target for developing novel insulin resistance agents.

Another approach to reducing the 4-HNE levels and ultimately the protein carbonylations is to develop better nucleophilic scavengers of 4-HNE. As a proof of concept, for this approach, S-adenosylmethionine, lysine, and histidine have been used as a supplement to mitigate insulin resistance. Therefore, the nucleophilic analogs with better capability to scavenge 4-HNE will reduce GLUT4 carbonylations and improve glucose tolerance.

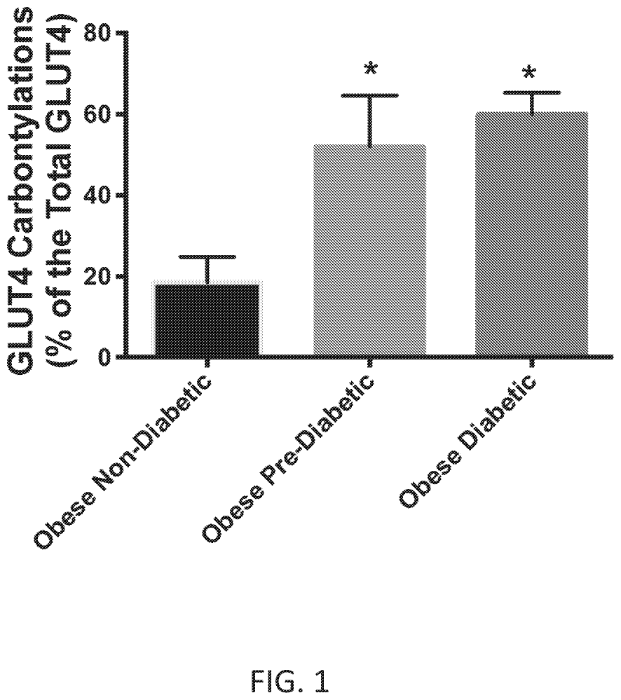

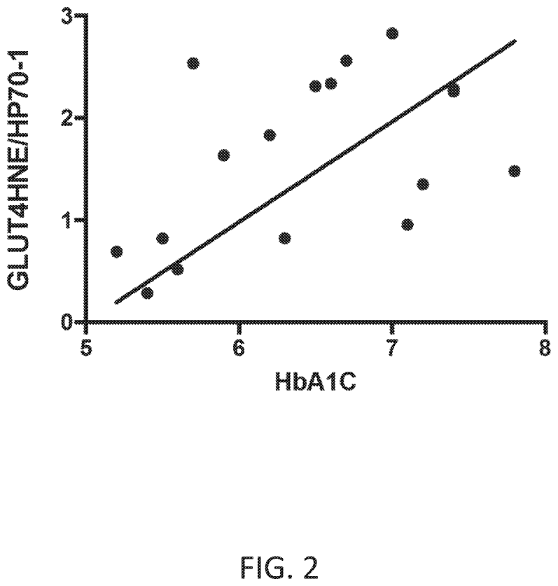

Results: Increased GLUT4 carbonylation is prevalent in pre-diabetes and diabetes and correlates with insulin resistance. Previous studies have shown that GLUT4 modification produces insulin resistance at the very beginning of excess caloric intake and weight gain. A comparison of the stoichiometry of GLUT4 modifications in adipose tissues from obese non-diabetic, pre-diabetic and diabetic subjects was performed to determine if the GLUT4 modification persist throughout obesity and type 2 diabetes. These types of analyses are more effectively studied using mass spectrometer based multiple reaction monitoring (MRM). This high throughput method does not require antibodies, is robust and is sensitive at sub-picomolar levels. The biological control for this study was HP70-1 because the levels of this protein were not altered in these subjects. Compared to obese non-diabetic, the percentage of GLUT4 K264 NHE-adduct to total GLUT4 was increased by approximately 2.5 folds in adipose tissue of pre-diabetic, and diabetic (see FIG. 1). Interestingly, about 50% GLUT4 carbonylations are identical to the ˜50% decrease in insulin-stimulated glucose uptake (GIR) after seven days of overnutrition. Interestingly, the GLUT4 carbonylations linearly increased with the insulin resistance marker HOMA-IR (FIG. 2). Collectively, these studies indicate that GLUT4 carbonylations are associated with insulin resistance and that GLUT4 carbonylation is a viable biomarker.

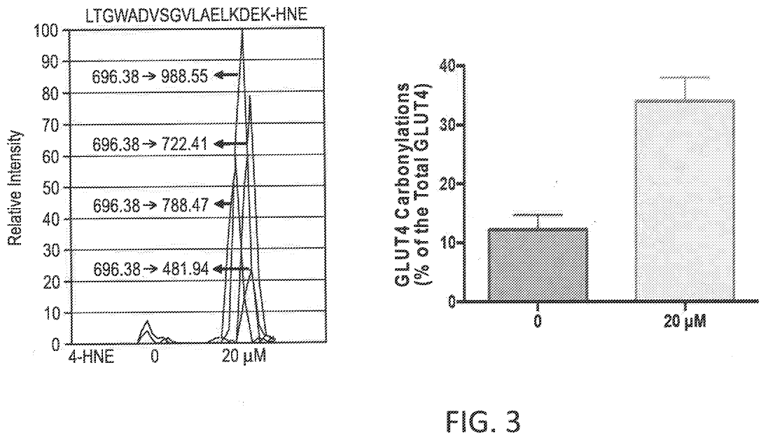

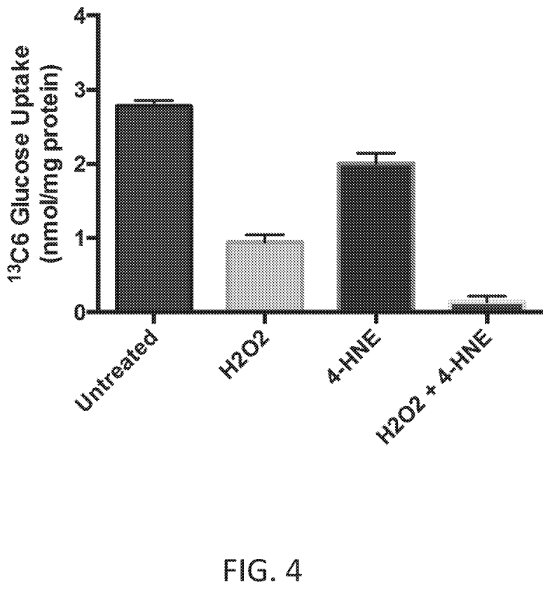

GLUT4 carbonylations impair its function. To directly link the function impairment due to GLUT4 carbonylations to insulin resistance in vitro, GLUT4-SNAP fusion construct were generated and retroviral transduction in 3T3-L1 adipocytes were performed to overexpress the GLUT4-SNAP protein. Twenty-four hours after the transduction, the cells were treated with and without 20 μM of 4-HNE for an additional 4h. This 4-HNE dose was chosen because it was similar to the physiological levels and was non-toxic. FIG. 3 demonstrates that 4-HNE (20 μM for 4 hr) induced the formation of the K264-HNE adduct in 3T3-L1 cells overexpressing GLUT4. 3T3-L1 adipocytes were treated with either 20 M of 4-HNE or H₂O₂ or the combination of both for 4 hr and were then stimulated with 100 nM insulin for 60 min. The glucose uptake was measured by an MRM method. FIG. 4 shows that the glucose uptake was reduced by 32% and 66% with 4-HNE and H₂O₂ treatment, respectively. The combination of both of the oxidative stress products resulted in 98% decrease in the glucose uptake.

1. A method of preventing or treating type 2 diabetes in a subject in need thereof comprising administering to the subject a therapeutically effective amount of a compound of Formula I, or a pharmaceutically acceptable salt thereof: 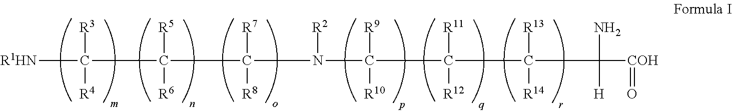
wherein:
R¹ is selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₈)alkyl, where the substituents on said substituted ara(C₁-C₆)alkyl and substituted heteroara(C₁-C₆)alkyl are selected from the group consisting of halogen, —CN, —NO₂, —NH₂, —NH(C₁-C₆)alkyl, —N[(C₁-C₆)alkyl)]₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
R² is selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
R³, R⁴, R⁷, R⁸, R⁹, R¹⁰, R¹³, and R¹⁴ are independently selected from the group consisting of hydrogen and —(C₁-C₆)alkyl;
R⁵ and R⁶ are independently selected from the group consisting of hydrogen, —(C₁-C₆)alkyl and —OH, provided that both R⁵ and R⁶ cannot be —OH;
R¹¹ and R¹² are independently selected from the group consisting of hydrogen, —(C₁-C₆)alkyl and —OH, provided that both R¹¹ and R¹² cannot be —OH;
m is 1, 2, 3 or 4;
n is 0, 1, 2, 3 or 4;
o is 0, 1, 2, 3 or 4;
p is 1, 2, 3 or 4;
q is 0, 1, 2, 3 or 4; and
r is 0, 1, 2, 3 or 4.

2. The method of claim 1, wherein a symptom of type 2 diabetes is treated.

3. The method of claim 2, wherein the symptom is selected from the group consisting of increased A1C level, increased fasting blood glucose, impaired glucose tolerance increased blood pressure, and increased blood glucose level.

4. The method of claim 1, wherein the therapeutically effective amount of a compound of Formula I is about 1 mg/day to about 1,000 mg/day.

5. The method of claim 1, wherein the compound of Formula I is administered with one or more additional therapeutic agent.

6. A method of preventing or treating pre-diabetes in a subject in need thereof comprising administering to the subject a therapeutically effective amount of a compound of Formula I, or a pharmaceutically acceptable salt thereof: 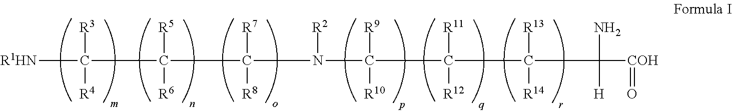
wherein:
R¹ is selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₈)alkyl and substituted heteroara(C₁-C₆)alkyl are selected from the group consisting of halogen, —CN, —NO₂, —NH₂, —NH(C₁-C₆)alkyl, —N[(C₁-C₆)alkyl)]₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHS₂NH₂;
R² is selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
R³, R⁴, R⁷, R⁸, R⁹, R¹⁰, R¹³, and R¹⁴ are independently selected from the group consisting of hydrogen and —(C₁-C₆)alkyl;
R⁵ and R⁶ are independently selected from the group consisting of hydrogen, —(C₁-C₆)alkyl and —OH, provided that both R⁵ and R⁶ cannot be —OH;
R¹¹ and R¹² are independently selected from the group consisting of hydrogen, —(C₁-C₆)alkyl and —OH, provided that both R¹¹ and R¹² cannot be —OH;
m is 1, 2, 3 or 4;
n is 0, 1, 2, 3 or 4;
o is 0, 1, 2, 3 or 4;
p is 1, 2, 3 or 4;
q is 0, 1, 2, 3 or 4; and
r is 0, 1, 2, 3 or 4.

7. The method of claim 6, wherein the subject has been diagnosed with impaired glucose tolerance, impaired fasting glucose, or insulin resistance.

8. The method of claim 6, wherein a risk of developing diabetes is reduced.

9. The method of claim 6, wherein a risk of developing a disease selected from the group consisting of kidney disease, diabetic retinopathy, heart attack and stroke, is reduced.

10. The method of claim 6, wherein a symptom of pre-diabetes is treated.

11. The method of claim 10, wherein the symptom is selected from the group consisting of increased A1C level, increased fasting blood glucose, impaired glucose tolerance, increased blood pressure, and increased blood glucose level.

12. The method of claim 6, wherein the therapeutically effective amount of a compound of Formula I is about 1 mg/day to about 1,000 mg/day

13. The method of claim 6, wherein the compound of Formula I is administered with one or more additional therapeutic agent.

14. A method of preventing or treating a condition in a subject in need thereof comprising administering to the subject a therapeutically effective amount of a compound of Formula I, or a pharmaceutically acceptable salt thereof: 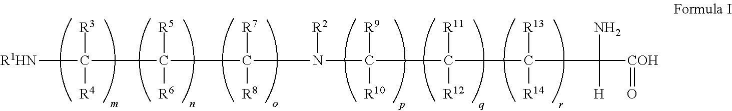
wherein:
R¹ is selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —NH(C₁-C₆)alkyl, —N[(C₁-C₆)alkyl)]₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
R² is selected from the group consisting of hydrogen, —(C₁-C₈)alkyl, —(C₁-C₈)alkenyl, —(C₁-C₈)alkynyl, unsubstituted or substituted -ara(C₁-C₆)alkyl, unsubstituted or substituted -heteroara(C₁-C₆)alkyl, where the substituents on said substituted ara(C₁-C₆)alkyl and substituted heteroara(C₁-C₆)alkyl are selected from the group consisting of halogen, —CN, —NO₂, —NH₂, —OH, halo(C₁-C₆)alkyl, —(C₁-C₆)alkoxy, halo(C₁-C₆)alkoxy, —SH, thio(C₁-C₆)alkyl, —SONH₂, —SO₂NH₂, —SO—(C₁-C₆)alkyl, —SO₂—(C₁-C₆)alkyl, —NHSO₂(C₁-C₆)alkyl, and —NHSO₂NH₂;
R³, R⁴, R⁷, R⁸, R⁹, R¹⁰, R¹³, and R¹⁴ are independently selected from the group consisting of hydrogen and —(C₁-C₆)alkyl;
R⁵ and R⁶ are independently selected from the group consisting of hydrogen, —(C₁-C₆)alkyl and —OH, provided that both R⁵ and R⁶ cannot be —OH;
R¹¹ and R¹² are independently selected from the group consisting of hydrogen, —(C₁-C₆)alkyl and —OH, provided that both R¹¹ and R¹² cannot be —OH;
m is 1, 2, 3 or 4;
n is 0, 1, 2, 3 or 4;
o is 0, 1, 2, 3 or 4;
p is 1, 2, 3 or 4;
q is 0, 1, 2, 3 or 4; and
r is 0, 1, 2, 3 or 4, and
wherein the condition is characterized by an increase in a measurement selected from the group consisting of A1C, glucose, insulin, homeostasis model of assessment of insulin resistance (HOMA-IR), oxidative stress in adipose tissue, and carbonylation of GLUT4.

15. The method of claim 14, wherein the subject has been diagnosed with impaired glucose tolerance, impaired fasting glucose, insulin resistance, pre-diabetes, or type 2 diabetes.

16. The method of claim 14, wherein a risk of developing pre-diabetes or type 2 diabetes is reduced.

17. The method of claim 13, wherein a risk of developing a disease selected from the group consisting of kidney disease, diabetic retinopathy, heart attack and stroke, is reduced.

18. The method of claim 14, wherein a symptom of the condition is treated.

19. The method of claim 18, wherein the symptom is selected from the group consisting of increased A1C level, increased fasting blood glucose, impaired glucose tolerance, increased blood pressure, and increased blood glucose level.

20. The method of claim 14, wherein the therapeutically effective amount of a compound of Formula I is about 1 mg/day to about 1,000 mg/day.

21. The method of claim 14, wherein the compound of Formula I is administered with one or more additional therapeutic agent.


SEQUENCE LISTING (1 sequence)

SEQ ID NO: 1 - protein, 18 aa, Artificial Sequence
  Adduct synthetically prepared
  MOD_RES (18)..(18): GLUT4-K264-NHE-adduct 4-NHE adduct linked to terminal lysine (corresponds to lysine 264 of GLUT4)
LTGWADVSGVLAELKDEK